SEQUENCE LISTING (58 sequences)

SEQ ID NO: 1 - protein, 7 aa, Artificial Sequence
  Description of Artificial Sequence non-naturally occurring peptide
LCFWKTC

SEQ ID NO: 2 - protein, 9 aa, Artificial Sequence
  Description of Artificial Sequence non-naturally occurring peptide
FCFWKTCFC

SEQ ID NO: 3 - protein, 8 aa, Artificial Sequence
  Description of Artificial Sequence non-naturally occurring peptide
CFWKTCSG

SEQ ID NO: 4 - protein, 7 aa, Artificial Sequence
  Description of Artificial Sequence non-naturally occurring peptide
FWKTSGG

SEQ ID NO: 5 - protein, 13 aa, Artificial Sequence
  Description of Artificial Sequence non-naturally occurring peptide
FWKTSTKTSTKWF

SEQ ID NO: 6 - protein, 11 aa, Homo sapiens
MFPNGTASSPS

SEQ ID NO: 7 - protein, 11 aa, Homo sapiens
QNGTLSEGQGS

SEQ ID NO: 8 - protein, 8 aa, Homo sapiens
AEQDDATV

SEQ ID NO: 9 - protein, 11 aa, Mouse
MFPNGTASSPS

SEQ ID NO: 10 - protein, 11 aa, Mouse
QNGTLSEGQGS

SEQ ID NO: 11 - protein, 8 aa, Mouse
AEQDDATV

SEQ ID NO: 12 - protein, 11 aa, Rat
MFPNGTAPSPT

SEQ ID NO: 13 - protein, 11 aa, Rat
QNGTLSEGQGS

SEQ ID NO: 14 - protein, 8 aa, Rat
AEQDDATV

SEQ ID NO: 15 - protein, 8 aa, Homo sapiens
MDMADEPL

SEQ ID NO: 16 - protein, 11 aa, Homo sapiens
QTEPYYDLTSN

SEQ ID NO: 17 - protein, 8 aa, Homo sapiens
QISPTPAL

SEQ ID NO: 18 - protein, 7 aa, Bovine
MDLVSEL

SEQ ID NO: 19 - protein, 11 aa, Bovine
QTEPYYDLASN

SEQ ID NO: 20 - protein, 8 aa, Bovine
AISPTPAL

SEQ ID NO: 21 - protein, 8 aa, Porcine
MDMAYELL

SEQ ID NO: 22 - protein, 11 aa, Porcine
QTEPYYDLTSN

SEQ ID NO: 23 - protein, 8 aa, Porcine
AISPTPAL

SEQ ID NO: 24 - protein, 8 aa, Mouse
MEMSSEQL

SEQ ID NO: 25 - protein, 11 aa, Mouse
QTEPYYDMTSN

SEQ ID NO: 26 - protein, 8 aa, Mouse
AISPTPAL

SEQ ID NO: 27 - protein, 8 aa, Rat
MELTSEQF

SEQ ID NO: 28 - protein, 11 aa, Rat
QTEPYYDMTSN

SEQ ID NO: 29 - protein, 8 aa, Rat
AISPTPAL

SEQ ID NO: 30 - protein, 7 aa, Homo sapiens
MDMLHPS

SEQ ID NO: 31 - protein, 11 aa, Homo sapiens
AGPSPAGLAVS

SEQ ID NO: 32 - protein, 8 aa, Homo sapiens
PLPEEPAF

SEQ ID NO: 33 - protein, 8 aa, Mouse
MATVTYPS

SEQ ID NO: 34 - protein, 11 aa, Mouse
AGTSLAGLAVS

SEQ ID NO: 35 - protein, 8 aa, Mouse
PLPEEPAF

SEQ ID NO: 36 - protein, 8 aa, Rat
MAAVTYPS

SEQ ID NO: 37 - protein, 11 aa, Rat
AGTSLAGLAVS

SEQ ID NO: 38 - protein, 8 aa, Rat
PLPEEPAF

SEQ ID NO: 39 - protein, 9 aa, Homo sapiens
MSAPSTLPP

SEQ ID NO: 40 - protein, 11 aa, Homo sapiens
GPGDARAAGMV

SEQ ID NO: 41 - protein, 7 aa, Homo sapiens
TSLDATV

SEQ ID NO: 42 - protein, 9 aa, Rat
MNTPATLPL

SEQ ID NO: 43 - protein, 10 aa, Rat
SDGTGTAGMV

SEQ ID NO: 44 - protein, 7 aa, Rat
TSLDATV

SEQ ID NO: 45 - protein, 7 aa, Homo sapiens
MEPLFPA

SEQ ID NO: 46 - protein, 10 aa, Homo sapiens
VGPAPSAGAR

SEQ ID NO: 47 - protein, 8 aa, Homo sapiens
ALPQEPAS

SEQ ID NO: 48 - protein, 7 aa, Rat
MEPLSLA

SEQ ID NO: 49 - protein, 10 aa, Rat
VGSASPMGAR

SEQ ID NO: 50 - protein, 8 aa, Rat
TLPEEPTS

SEQ ID NO: 51 - protein, 8 aa, Artificial Sequence
  Description of Artificial Sequence non-naturally occurring peptide
YDTKVFCS

SEQ ID NO: 52 - protein, 11 aa, Artificial Sequence
  Description of Artificial Sequence non-naturally occurring peptide
AYMGWSCTKWF

SEQ ID NO: 53 - protein, 16 aa, Artificial Sequence
  Description of Artificial Sequence non-naturally occurring peptide
FWKTSKHWSYGLRDGC

SEQ ID NO: 54 - protein, 12 aa, Rat
FRCPPCTERLAA

SEQ ID NO: 55 - protein, 12 aa, Rat
EVLFRCPPCTPE

SEQ ID NO: 56 - protein, 10 aa, Rat
GAGAVGAPVV

SEQ ID NO: 57 - protein, 12 aa, Rat
DEAIHCPPCSEE

SEQ ID NO: 58 - protein, 4 aa, Artificial Sequence
  Description of Artificial Sequence non-naturally occurring peptide
FWKT

(19) United States
(12) Patent Application Publication
(10) Pub. No.: US 2002/0187925 A1
(43) Pub. Date: Dec. 12, 2002

(54) Modulating the activity of hormones or their receptors - peptides, antibodies, vaccines and uses thereof

(76) Inventors: David J. Kingston, Glen Waverley (AU); Norman L. Gerraty, Mount Eliza (AU); Simon L. Westbrook, Balwyn (AU)

Correspondence Address: Stephen A. Bent, FOLEY & LARDNER, Washington Harbour, 3000 K Street, N.W., Suite 500, Washington, DC, 20007-5109, US

(21) Appl. No.: 09/758,198

(22) Filed: Jan. 12, 2001

Related U.S. Application Data
(62) Division of application No. 09/194,218, filed on Feb. 5, 1999, now abandoned.
Application No. 09/194,218 is a 371 of international application No. PCT/AU97/00312, filed on May 22, 1997.

(30) Foreign Application Priority Data
May 22, 1996 (AU) PN9990

(51) Int. Cl. C07K 1/00; C07K 14/00; C07K 17/00

(52) U.S. Cl. 514/2; 424/554; 530/350; 530/399

(57) ABSTRACT

This invention relates to immunogenic, non-naturally occurring peptides and immunologically reactive molecules thereto which modulate the activity of hormones or the receptors therefor. Methods of modulating hormonal activity in an animal and compositions therefor are also contemplated.

### FIELD OF THE INVENTION

The present invention relates to peptides, immunologically reactive molecules (IRM) specific for the peptides, pharmaceutical compositions including vaccines incorporating the peptides or IRM and to uses of these in animals.

### BACKGROUND

Over the past three decades alternative methods, other than genetic selection and dietary manipulation, have been sought to improve/modulate the efficiency of animal production; for example liveweight gain, improved efficiency of utilisation of feed, milk yield, wool production, survival rate and body composition. The techniques implemented to achieve such outcomes have been based upon the administration of exogenous hormones and/or the production of transgenic animals.

Animal welfare issues and increasing consumer and government antipathy towards the use of exogenous hormone treatments and transgenic technology have led researchers to explore alternative “drug free” methods of improving the efficiency of animal production. Over the past decade attention has been focused on the possibility of utilising the immune response to manipulate the endocrine system.

There are examples reported in the scientific literature of antibody-mediated enhancement or suppression of growth, body composition, appetite, and reproductive physiology. These include luteinizing hormone (LH), thyroid-stimulating hormone (TSH; Melmed et al., 1980), inhibin (Scanlon et al., 1993), growth hormone (Pell and James, 1995), insulin like growth factor (Pell and Aston, 1995), prolactin (Lindstedt, 1994), growth hormone releasing factor (Moore et al., 1992), vasopressin (Kamoi et al., 1977), somatostatin (Westbrook et al., 1993), gastrin (Dockray and Taylor, 1976), cholecystokinin (CCK; Pekas and Trout, 1993), testosterone (Thomson et al., 1985), progesterone (Kaushansky et al., 1977), prostaglandin F_{2α} (PGF_{2α}; Crowe et al., 1995), and adrenocorticotropin (ACTH; Wynn et al., 1995).

The reasons for attempting to elicit immune responses to “self” hormones are varied. These include efforts to improve the production of farm animals; by increasing meat quality, reducing back-fat, promoting appetite, enhancing liveweight gain, improving milk production, altering the constituents of milk, and manipulating fertility. In addition there has been interest in easing labour requirements associated with farm management, overcoming disease and gaining a greater understanding of endocrine and autocrine interactions.

Of all the peptide, protein and steroid antigens listed above, only two commercial preparations based on immunological manipulation of endogenous hormones appear to be available. These are Fecundin®, an androstenedione antigen, which results in an improved fecundity (Scaramuzzi et al, 1977), and Vaxtrate®, a LHRH antigen, which induces immuno-neutralisation of LHRH with resulting inhibition of steroidogenesis in the ovaries and testes of cattle (Hoskinson et al., 1990).

### SUMMARY OF THE INVENTION

In work leading up to the invention the inventors produced a number of peptides based on native hormones, or receptors of native hormones and administered these to animals. Administration of the peptides together with an adjuvant resulted in a number of economically significant effects in the animals such as improved live weight gain, improved milk production, improved wool production, meat quality, efficiency of food utilisation and the like.

Thus the invention provides a series of peptides, of various amino acid sequences which are capable of eliciting specific antibodies that alter an array of physiological functions in animals. These physiological functions include digestion, nutrient uptake and metabolism of absorbed substrates, which result in improved productive capacities of immunised animals. Particularly notable are the modifications of the gastrointestinal tract resulting in improved digestion and associated benefits.

In a first aspect the present invention provides a non-naturally occurring peptide with an amino acid sequence which is derived from, or is similar to a native animal hormone, carrier protein, binding protein or receptor for said hormone, wherein said peptide is capable of eliciting one or more antibodies which are able to modulate the activity of said hormone or receptor in vivo.

The term “non-naturally occurring” means that the peptide is not the same as that produced by protein synthesis in nature but is produced by the human hand. Peptides may be produced by standard peptide synthesis techniques, or by recombinant DNA techniques or the like.

The term “peptide” refers to any molecule formed at least partly of amino acids wherein the amino acids are joined by peptide bonds. The peptides may be made up of only a few amino acids or may be polypeptides. Therefore, proteins and microproteins are also encompassed by this term. The amino acids comprising the peptides may be naturally occurring or synthetic amino acids. The peptides may be essentially amino acid sequences, and may also comprise non-amino acid components such as carbohydrates or fatty acids, or comprise non-natural amino acid-like structures.

The term “amino acid sequence derived from, or is similar to” used above refers to the fact that the amino acid sequence may be based on a native sequence or be similar to a native sequence. The term “derived from” does not indicate the actual origin of the peptide (as it may be synthetic or recombinant in origin) but indicates a peptide at least partly homologous to the native hormone or receptor. The peptide is of course different to the native hormone or receptor in that it may comprise a fragment thereof or two or more non-contiguous fragments thereof. It may also comprise other amino acids not present in the native hormone or receptor.

The term “native animal hormone, carrier protein, binding protein or receptor of the hormone” refers to hormones, carrier proteins, binding proteins or receptors which occur in animals. This may be any type of animal but is preferably a vertebrate, more preferably a mammal.

The term “capable of eliciting one or more antibodies” refers to the ability of the peptide to elicit or stimulate what appears to be primarily an antibody mediated immune response. There may be some cell mediated immunity involved also. The peptide may not be able to stimulate an immune response by itself especially in the case of small peptides which may need to be present on a carrier molecule. Nonetheless such a peptide still falls within the definition of “capable of eliciting one or more antibodies”.

The term “able to modulate the activity of said hormone or receptor in vivo” refers to the ability of the antibody to alter, adjust or vary the activity of the hormone or its receptor in a live animal. The alteration, adjustment or variation will generally be in the form of a down regulation or inhibition of the hormone or receptor although other effects such as an increase in hormone or receptor activity are also contemplated.

Preferably the peptide is not biologically active. Although it is based on a native hormone or receptor, preferably the peptide does not have the biological activity of that hormone or receptor.

The peptide may be based on any hormone or receptor involved in regulating physiological functions.

Preferably the peptides are able to elicit antibodies to the following hormones or hormone receptors: somatostatin, glucagon, gastrin, cholecystokinin, somatostatin receptors, insulin-like growth factor binding proteins (IGFBP). In one embodiment, the peptides elicit antibodies to hormones of the reproductive tract, in particular luteinising hormone releasing hormone (LHRH), hormones of the adrenal gland, such as adrenal corticotropic hormone (ACTH), or of the stomach such as gastrin and cholecystokinin.

The inventors have produced a number of peptides based on portions of somatostatin (Patel 1992), somatostatin receptors (SSTR) (Resine & Bell, 1995) and insulin-like growth factor binding protein 1 to 4 (IGFBP) (Cohick & Clemmons, 1993).

In a particularly preferred aspect the invention provides a non-naturally occurring peptide with an amino acid sequence based on that of somatostatin (SRIF). The preferred sequence produces antibodies which preferentially target SSTR2, SSTR3 and SSTR5, in contrast to somatostatin which produces antibodies that bind to the receptor comprising SSTR1 to 6.

The peptide of the invention is able to increase levels of circulating insulin, IGF-I and IGF-II and, increase levels of anabolism, decrease gastric activity and/or improve digestion in an animal.

More particularly the invention provides a peptide having the following sequence as represented by the single letter amino acid code:

LCFWKTC (SEQ ID NO: 1)

This peptide binds to and produces antibodies which exhibit an affinity for SSTR2, SSTR3 and SSTR5 of the somatostatin receptor. Without wishing to be bound by any proposed mechanism for the observed advantages, it appears that the sequence F-W-K-T is the key to blocking of SSTR2, SSTR3 and SSTR5.

The invention also relates to a derivative or variant of SEQ ID NO:1 in which the size and the shape of the exposed rings of the antigen are the smallest diameter possible.

SEQ ID NO:1 is a relatively soluble peptide in sterile physiological saline.

Preferably, the peptide is presented as a cyclised sequence, e.g.:

F-C-F-W-K-T-C-F-C (SEQ ID NO:2) or

C-F-W-K-T-C-S-G (SEQ ID NO:3)

The peptide comprising the core sequence FWKT may also be cyclised by means other than inclusion of cysteine residues which form disulphide bonds. A particularly preferred form of the cyclic peptide of the invention is one wherein the linking group or sequence is as short as possible, and the structural configuration is one which allows the portion of the molecule comprising FWKT to bind to the somatostatin receptor. In a more particularly preferred embodiment, the spatial configuration corresponding to the sequence FWKT in the cyclic peptide is that it occupies minimal space. Ideally, the arc in the cyclic peptide that is formed by the sequence FWKT is as small as possible and complements its target receptor.

A cyclic peptide which stimulates production of antibodies with an affinity for SSTR2, 3 and 5 is particularly preferred.

The peptide may also be presented as a linear peptide singly, e.g.:

F-W-K-T-S-G-G (SEQ ID NO:4) or as a dimer, e.g.:

F-W-K-T-S-T-K-T-S-T-K-W-F (SEQ ID NO:5)

In yet another embodiment, the invention provides an immunogenic protein or molecule which comprises a sequence which produces antibodies which have a particular affinity for SSTR2, 3 and 5. The protein or molecule may be of any type as long as the binding of antibodies to the receptors is stimulated.

In another preferred aspect the invention provides a non-naturally occurring peptide with an amino acid sequence which is at least partly homologous to a native animal hormone receptor, wherein said peptide is capable of eliciting one of more antibodies which are able to modulate the activity of said receptor in vivo

In a particularly preferred aspect the invention provides a non-naturally occurring peptide with an amino acid sequence at least partly homologous to that of a somatostatin receptor (SSTR). Such peptides have the ability to increase at least one biological activity selected from the group consisting of weight gain, birth weight, growth rates, milk production, levels of circulating insulin, IGF-I and IGF-II, fibre production, milk production, and muscle weight.

More particularly preferred is a peptide having an amino acid sequence homologous to amino acid residues 1 to 11, 30-57 and/or between the sixth and seventh transmembrane domain of SSTR. The transmembrane domain in question has been reported to occur at residues 274 to 305. The amino acid positions for SSTR are relative to the NH₃⁺ terminal of the SSTR for all vertebrate species. Preferably the peptide is based on SSTR from humans, pigs, cattle, mice or rats. The peptides may be based on SSTRs from other species such as sheep, goats, camels, llamas, alpacas, chickens, ducks, turkeys, ostriches, emus and fish.

Still more particularly the invention provides a peptide having the following sequence as represented by the single letter amino acid code, and derivatives and variants thereof:

| SEQ | | | |
|---|---|---|---|
| ID NO | Peptide | Based on | Residue No. |
| 6 | MFPNGTASSPS | Human SSTR 1 | 1-11 |
| 7 | QNGTLSEGQGS | Human SSTR 1 | 47-57 |
| 8 | AEQDDATV | Human SSTR 1 | 297-305 |
| 9 | MFPNGTASSPS | Mouse SSTR 1 | 1-11 |
| 10 | QNGTLSEGQGS | Mouse SSTR 1 | 47-57 |
| 11 | AEQDDATV | Mouse SSTR 1 | 297-305 |
| 12 | MFPNGTAPSPT | Rat SSTR 1 | 1-11 |
| 13 | QNGTLSEGQGS | Rat SSTR 1 | 47-57 |
| 14 | AEQDDATV | Rat SSTR 1 | 297-305 |
| 15 | MDMADEPL | Human SSTR 2 | 1-8 |
| 16 | QTEPYYDLTSN | Human SSTR 2 | 32-42 |
| 17 | AISPTPAL | Human SSTR 2 | 282-290 |
| 18 | MDLVSEL | Bovine SSTR 2 | 1-7 |
| 19 | QTEPYYDLASN | Bovine SSTR 2 | 31-41 |
| 20 | AISPTPAL | Bovine SSTR 2 | 281-289 |
| 21 | MDMAYELL | Porcine SSTR 2 | 1-8 |
| 22 | QTEPYYDLTSN | Porcine SSTR 2 | 32-42 |
| 23 | AISPTPAL | Porcine SSTR 2 | 282-290 |
| 24 | MEMSSEQL | Mouse SSTR 2 | 1-8 |
| 25 | QTEPYYDMTSN | Mouse SSTR 2 | 31-41 |
| 26 | AISPTPAL | Mouse SSTR 2 | 282-290 |
| 27 | MELTSEQF | Rat SSTR 2 | 1-8 |
| 28 | QTEPYYDMTSN | Rat SSTR 2 | 30-40 |
| 29 | AISPTPAL | Rat SSTR 2 | 283-291 |
| 30 | MDMLHPS | Human SSTR 31-7 | 1-7 |
| 31 | AGPSPAGLAVS | Human SSTR 3 | 31-41 |
| 32 | PLPEEPAF | Human SSTR 3 | 283-291 |
| 33 | MATVTYPS | Mouse SSTR 3 | 1-8 |
| 34 | AGTSLAGLAVS | Mouse SSTR 3 | 32-42 |
| 35 | PLPEEPAF | Mouse SSTR 3 | 284-292 |
| 36 | MAAVTYPS | Rat SSTR 3 | 1-8 |
| 37 | AGTSLAGLAVS | Rat SSTR 3 | 32-42 |
| 38 | PLPEEPAF | Rat SSTR 3 | 284-292 |
| 39 | MSAPSTLPP | Human SSTR 4 | 1-9 |
| 40 | GPGDARAAGMV | Human SSTR 4 | 31-41 |
| 41 | TSLDATV | Human SSTR 4 | 282-290 |
| 42 | MNTPATLPL | Rat SSTR 4 | 1-9 |
| 43 | SDGTGTAGMV | Rat SSTR 4 | 31-41 |
| 44 | TSLDATV | Rat SSTR 4 | 282-290 |
| 45 | MEPLFPA | Human SSTR 5 | 1-7 |
| 46 | VGPAPSAGAR | Human SSTR 5 | 30-40 |
| 47 | ALPQEPAS | Human SSTR 5 | 282-290 |
| 48 | MEPLSLA | Rat SSTR 5 | 1-7 |
| 49 | VGSASPMGAR | Rat SSTR 5 | 33-43 |
| 50 | TLPEEPTS | Rat SSTR 5 | 283-291 |

The term “derivatives and variants” thereof refers to peptides with different amino acid sequences having substantially the same or similar antigenic activity. Such derivatives or variants may have amino acid substitutions, insertions or deletions compared to the preferred sequences listed above. Typical substitutions are those made in accordance with Table 1 below.

TABLE 1
Suitable residues for amino acid substitutions

| Original Residue | Exemplary Substitutions |
|---|---|
| Ala | Ser |
| Arg | Lys |
| Asn | Gln; His |
| Asp | Glu |
| Cys | Ser |
| Gln | Asn |
| Glu | Ala |
| Gly | Pro |
| His | Asn; Gln |
| Ile | Leu; Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Where the peptide is derivatised by amino acid substitution, the amino acids are generally replaced by other amino acids having like properties such as hydrophobicity, hydrophilicity, electronegativity, bulky side chains and the like. Amino acid substitutions are typically of single residues. Amino acid insertions will usually be in the order of about 1-10 amino acid residues and deletions will range from about 1-20 residues. Preferably, deletions or insertions are made in adjacent pairs, i.e. a deletion of two residues or insertion of two residues.

For example, the peptide of the invention in the cyclised or cyclic form may be varied by substitution of some of the key amino acids with similar amino acids, or amino acid-like structures. A particularly preferred sequence is:

NMe-Y-D-T-K-V-F-C-S (SEQ ID NO:51) although this sequence is very poorly soluble in sterile saline solutions.

Piglets suckling dams which had been vaccinated with this molecule in the last trimester of pregnancy grew an average of 40% faster from birth to weaning than did corresponding piglets which were suckling non-immunised dams.

The peptides and immunologically active molecules of the invention may be administered as the sole active agent, or be co-administered with one or more other agents. For example, SEQ ID NO:1 can be co-administered with gastrin and/or cholecystokinin, together with a suitable carrier.

In one embodiment, sequences such as SEQ ID NO:2 to 5 may be co-presented as separate peptides or as one peptide molecule comprising sequences specifically targeting other hormones such as gastric hormones. An example of such a sequence is:

A-Y-M-G-W-S-C-T-K-W-F (SEQ ID NO:52)

When this antigen was injected three times into growing lambs with the preferred delivery vehicle (oil), anti-SRIF and anti-cholecystokinin antibodies were detectable at 84 days of age. By this time, the immunised lambs had grown an average of 20% more than non-immunised lambs.

Reproductive hormones may also be targeted and an example of a sequence for co-administration with the peptide preferred for this invention is:

F-W-K-T-S-K-H-W-S-Y-G-L-R-D-G-C (SEQ ID NO:53)

Male pigs immunised with this peptide three times from 12 weeks of age grew an average of 12 weeks faster than non-immunised pigs to 24 weeks of age, and the testicle size of the immunised pigs was approximately 50% the size of those of the non-immunised animals.

In a particularly preferred aspect the invention provides a non-naturally occurring peptide with an amino acid sequence at least partly homologous to insulin-like growth factor binding protein (IGFBP). Such peptides are able to modulate carbohydrate metabolism and thereby improve growth of animals. The peptides may also be useful in the prevention or treatment of diabetes.

More particularly preferred is a peptide which includes in its sequence a portion of a native IGFBP which binds insulin-like growth factor, preferably at least some of the region of residues 1 to 10 or 1 to 13 of a native IGFBP. The amino acid residues are relative to the NH₃⁺ terminal of IGFBP. Preferably the peptide is based on IGFBP from humans, pigs, cattle, mice or rats. The peptide may also be based on IGFBPs from other species such as sheep, goats, camels, llamas, alpacas, chickens, ducks, geese, turkeys, ostriches, emus and fish.

Still more particularly the invention provides a peptide having one of the following sequences as represented by the single letter amino acid code, or a derivative or variant thereof.

| SEQ ID | | | Residue |
|---|---|---|---|
| NO. | Peptide | Based on | No. |
| 54 | FRCPPCTERLAA | Rat IGFBP | 11-12 |
| 55 | EVLFRCPPCTPE | Rat IGFBP 2 | 1-12 |
| 56 | GAGAVGAPVV | Rat IGFBP 3 | 1-12 |
| 57 | DEAIHCPPCSEE | Rat IGFBP 4 | 1-12 |

The peptides of the invention may be referred to herein as “peptides based on SSTR or peptides based on IGFBP”, for example. Peptides may be based on one or more hormones, carrier proteins or hormone receptors.

The peptides contemplated herein may be chemically synthesised, for example by solid phase peptide synthesis or may be prepared by subjecting the native peptides to hydrolysis or other chemically disruptive processes to produce fragments of the molecule. Alternatively, the peptides may be made by in vitro or in vivo recombinant DNA synthesis. In this case, the peptides may need to be synthesised in combination with other proteins and then subsequently isolated by chemical or enzymic cleavage, or the peptides or polyvalent peptides may be synthesised in multiple repeat units. The selection of a particular method of producing the subject peptides will depend on factors such as the required type, quantity and purity of the peptides as well as ease of production and convenience.

Preferably the peptides of the invention are at least partially purified. More preferably the peptides are in a substantially purified form.

In a second aspect the invention provides an immunologically reactive molecule (IRM) which is specific for the peptide of the first aspect of the invention.

The term “immunologically reactive molecule” refers to a molecule which is able to bind to another molecule, such as an antigen or a peptide capable of functioning as an antigen when present on a carrier. Immunologically reactive molecules are typically antibodies including naturally occurring antibodies, recombinant antibodies, scantibodies, synthetic antibodies including fusions or chimeras of antibodies, and functional fragments of any of the foregoing, such as Fab and F(ab′)₂. Where the antibody IRM is a recombinant form, the molecule may be encoded by a naturally occurring or synthetic nucleotide sequence and expressed in any convenient expression system. Where the molecule is synthetic, it is conveniently prepared by the step-wise addition of single amino acid groups or amino acid fragments of antibodies. With regard to the latter, the synthetic antibody may be a fusion or chimeric antibody comprising light or heavy chains derived from other antibodies.

Antibodies and other IRMs of the present invention may be of any animal origin, including from mammals such as humans, livestock animals, companion animals, wild animals and laboratory test animals (eg. mice, rats, rabbits and guinea pigs). An “animal” antibody also extends to an antibody from non-mammalian species such as birds, eg. chickens and other poultry, emus and ostriches.

Binding of the peptide or IRM may occur in the target hormone receptor site (usually by the use of biologically active antigenic sequences), or to areas of the cell membrane adjacent to the targeted receptor site, in which case the antigenic fragment has no biological activity.

In a third aspect the present invention provides a peptide of the first aspect of the invention coupled to a suitable carrier such that a peptide/carrier complex is formed.

The peptide/carrier complex is particularly advantageous where the peptide is relatively small (MW less than 10,000) and not particularly immunogenic when administered alone.

Suitable carriers are generally large molecules which are capable of coupling with the peptide. In order to elicit an immune response, the peptides generally need to be coupled to carrier proteins, to make the antigen significantly “foreign” and to increase the molecular weight of the antigen. In general, it appears that the more foreign or the more immunogenic a carrier protein is to the vaccine recipient the greater the antibody response is likely to be (Meloen, 1995) Large protein molecules, notably keyhole limpet haemocyanin, bovine serum albumin, bacterial toxins, ovalbumin and thyroglobulin (see Meloen, 1995), are used generally for conjugation to small molecular weight antigens; however a multiple antigen presentation (MAP) system, for example poly-L-lysine, is preferred.

In a fourth aspect the present invention provides a pharmaceutical composition comprising an immunogenically effective amount of the peptide of the first aspect of the invention, or an amount of the IRM of the second aspect of the invention sufficient to confer passive immunity, together with a pharmaceutically or veterinarily acceptable carrier or excipient, and optionally comprising an adjuvant.

In the following description, the peptide of the present invention is referred to as the “active ingredient”.

The active ingredient of the pharmaceutical composition is contemplated to exhibit excellent activity in stimulating, enhancing or otherwise facilitating a humoral immune response in animals when administered in an amount which depends on the particular case. For example, for about 0.5 μg to about 20 mg of protein which may be considered per kilogram of body weight per day may be administered in one or more of daily, weekly or monthly or in other suitable time intervals or the dose may be proportionally reduced as indicated by the exigencies of the situation. The active compound may be administered by injection in a convenient manner or via a genetic sequence in a viral or bacterial vector.

The active ingredient may also be administered in dispersions prepared in sterile physiological saline, glycerol, liquid polyethylene glycols, and/or mixtures thereof and in oils. Under ordinary conditions of storage and use, these preparations contain a preservative to prevent the growth of microorganisms.

The pharmaceutical forms suitable for in vivo administration include sterile aqueous solutions (where water soluble) or dispersions and sterile powders for the extemporaneous preparation of sterile injectable solutions or dispersion. In all cases the form must be sterile and must be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thormerosal and the like. In many cases, it will be preferable to include isotonic agents, for example, sugars or sodium chloride, or gelling agents such as cyclodextrins, gelatin, alginate, and the like. Prolonged absorption of the injectable compositions can be brought about by the use in the compositions of agents delaying absorption, for example.

Sterile injectable solutions are prepared by incorporating the active ingredient in the required amount in the appropriate solvent with various of the other ingredients enumerated above, as required, followed by filtered sterilisation. Generally, dispersions are prepared by incorporating the various sterilised active ingredient(s) into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

As used herein “pharmaceutically or veterinarily acceptable carriers and/or diluents” include any and all solvents, dispersion media, aqueous solutions, coatings, antibacterial and antifungal agents, isotonic and absorption delaying agents, and the like. The use of such media and agents for pharmaceutically or veterinarily active substances is well known in the art. Except insofar as any conventional media or agent is incompatible with the active ingredient, use thereof in the composition is contemplated. Supplementary active ingredients can also be incorporated into the compositions.

Preferably the pharmaceutical composition comprises the protein coupled to a carrier. More preferably the pharmaceutical composition is in the form of a vaccine preparation.

Many substances may be used to deliver the peptide or molecules of the invention. High levels of circulatory humoral antibodies to SRIF have been generated following immunisation with SRIF antigens in physiological saline, Freunds complete adjuvant (FCA), muramyl dipeptide (MDP), Freunds incomplete adjuvant (FIA & MDP), DEAE-dextran, and Quill A.

The invention also provides a novel delivery vehicle for the peptide of the invention, comprising an oil derived from a deep sea shark. Thus, in a fifth aspect, the invention provides a veterinarily or pharmaceutically acceptable carrier, comprising shark oil which has immune adjuvant activity.

The oil is desirably of a type which stimulates antibody production in epithelial surfaces of the lung, the respiratory, gastrointestinal or urogenital tract, or the like. In a particularly preferred embodiment, the oil enhances antibody secretion in the mucosa of the mammary gland, thereby producing immuno-active colostrum or milk.

The oil typically comprises the following components:

| Hydrocarbons | nil-2% |
|---|---|
| Wax esters | nil-2% |
| Free fatty acids | less than 2% |
| Polar lipids | 10-15% |
| Diacylglycerol ethers | 30-50% |
| Triacyglycerols | 40-70% |

Approximately 80% of the fatty acids present are in the monounsaturated form.

The carbon chain length and proportion of the fatty acids of the typical oil are as follows:

Typical analysis

| | |
|---|---|
| C14 | 1%-2% |
| C15 | >1% |
| C16 | 18%-20% |
| C17 | 1%-4% |
| C18 | 42%-65% |
| C19 | 0.1%-2% |
| C20 | 5%-15% |
| C21 | >1% |
| C22 | 0.1%-18% |
| C23 | nil |
| C24 | 0%-5% |

The oil is preferably alkoxyglycerol-rich in that it comprises a triacylglycerol with the general formula CH₂OH.CHOH.CH₂OR, where R is a long chain radical, primarily and preferably C₁₆ and C₁₈.

Within this general structure, the following glycerol ethers are especially preferred:

| | |
|---|---|
| 20-70% | octadec-9-enylglyceryl ether |
| 3-25% | hexadecylglyceryl ether |
| 1-15% | hexadec-7-enylglyceryl ether |
| 1.5-20% | octadecyl glyceryl ether |
| 1-15% | eicosa-9-enylglyceryl ether |

The following are preferably added to this oil:

| | |
|---|---|
| 1-25% | lecithin |
| 1-25% | DL alpha tocopherol acetate |
| 0-3% | 1,2,5-dihydroxycholecalciferol |
| 0-5% | vitamin A |
| 0-40% | non-mineral oil (typically having a |
| | triacylglycerol structure) |

In a sixth aspect the invention provides a method of producing an immunogenic composition, comprising the steps of contacting a peptide capable of eliciting an immune response with an oil and bringing said peptide and oil into a form suitable for administration, wherein said oil comprises 30-50% diacylglycerol-ethers. Preferably, the ethers comprise:

| | |
|---|---|
| 20-70% | octadec-9-enylglyceryl ether |
| 3-25% | 1-hexadecylglyceryl ether |
| 1-15% | hexadec-7-enylglyceryl ether |
| 1.5-20% | octadecyl glyceryl ether |
| 1-15% | eicosa-9-enylglyceryl ether |
| 1-25% | lecithin as the emulsifying agent |
| 1-25% | DL alpha tocopherol acetate |
| 0-3% | 1,2,5-dihydroxycholecalciferol |
| 0-5% | vitamin A |
| 0-40% | non-mineral oil |

In a particularly preferred embodiment, the immunogenic composition comprises peptide No. 1 emulsified with the oil (“FEEDMIZA”).

The term “immunogenic composition” refers to a pharmaceutical or veterinary composition which is able to elicit an immune response. This includes vaccines and the like. The composition may be formulated for various forms of administration such as injection (intraperitoneal, subcutaneous, intramuscular or intramammary), orally, nasal spray, skin patch or the like.

The term “a peptide capable of eliciting an immune response” refers to any peptide which is immunogenic per se or is capable of inducing an immune response once administered in the composition. Preferably the peptide of the first aspect of the invention is used in the composition, but other peptides, such as bacterial and viral antigens, are also contemplated.

Those skilled in the art will be familiar with methods which may be employed to produce the immunogenic composition. The composition may include other components such as other active ingredients, drugs or adjuvant as desired.

In a seventh aspect the present invention provides a method of delivering to an animal a peptide capable of eliciting an immune response, comprising the step of administering said peptide together with an effective amount of an oil comprising:

| | |
|---|---|
| 20-70% | octadec-9-enylglyceryl ether |
| 3-25% | 1-hexadecylglyceryl ether |
| 1-15% | hexadec-7-enylglyceryl ether |
| 1.5-20% | octadecyl glyceryl ether |
| 1-15% | eicosa-9-enylglyceryl ether |
| 1-25% | lecithin |
| 1-25% | DL alpha tocopherol acetate |
| 0-3% | 1,2,5-dihydroxycholecalciferol |
| 0-5% | vitamin A |
| 0-40% | non-mineral oil |

The peptide is preferably emulsified with an effective amount of the oil.

The term “an effective amount of oil” refers to an amount of oil which will be effective in enabling the peptide to elicit an immune response, particularly where the peptide is not immunogenic by itself or only induces low levels of immunity. Such an amount will generally be about 50 to 80% of the total volume of the composition, preferably 60-70% of the total volume, more preferably about 66 to 67% of the total volume of the composition.

In an eighth aspect the present invention provides a method of modulating one or more hormonal responses in an animal, comprising the step of administering to said animal a hormone-modulating effective amount of the peptide of the first aspect of the invention or the IRM of the second aspect of the invention.

The hormonal responses include endocrine and/or paracrine responses.

The term “modulating one or more hormonal responses” refers to altering, adjusting or varying the hormonal responses in the animal concerned.

The animal may be any animal, preferably a vertebrate, more preferably a mammal. The term animal includes humans, ruminants, birds and reptiles. Preferably the animal is a domestic or production animal such as a pig, goat, camel, sheep, alpaca, llama, chicken, goose, duck, turkey, ostrich, emu, fish or other economically important animal.

Preferably the peptide administered is the peptide complex discussed above, preferably having more than one antigen. More preferably the peptide is administered to the animal in the form of a pharmaceutical preparation, still more preferably, a vaccine formulation.

In a preferred aspect the invention relates to a method of modulating the hormonal responses to one or more of somatostatin, gastrin, insulin, glucagon, prolactin, molitin, cholecystokinin, secretin, prostaglandins, IGF-I, IGF-II, growth hormone and thyroid hormones by administration of the peptide or IRM of the invention.

In another preferred aspect the invention relates to a method of enhancing gastrointestinal function in an animal, comprising the step of administering an effective amount of a peptide based on SSTR and/or IGFBP to said animal.

The term “enhancing gastrointestinal function” refers to promoting digestion of and absorption of key metabolic substrates.

In another preferred aspect the invention provides a method of increasing anabolism and/or body weight in an animal, comprising the step of administering an effective amount of a peptide based on SSTR and/or IGFBP to said animal.

Preferably the peptide is that of the invention described earlier.

In another preferred aspect the invention provides a method of increasing circulating insulin, IGF-I and/or IGF-III in an animal, comprising the step of administering an effective amount of a peptide based on SSTR and/or IGFBP to said animal.

In another preferred aspect the invention provides a method of suppressing gastric enzymes in an animal, comprising the step of administering an effective amount of a peptide based on SSTR to said animal.

Preferably the peptide is that of the invention described earlier. More preferably the peptide is that based on SSTR.

In another preferred aspect the invention provides a method of increasing fibre production and optionally further altering the proportion of secondary to primary follicles in a fibre-producing animal, comprising the step of administering an effective amount of a peptide based on SSTR and/or IGFBP to said animal.

Preferably the peptide is that of the invention described earlier.

The term “a fibre producing animal” refers to any animal which produces a useful fibre such as wool or the like and includes sheep, goats, llamas and alpacas.

In another preferred aspect the invention provides a method of increasing milk production in a milk-producing animal, comprising the step of administering an effective amount of a peptide based on SSTR and/or IGFBP to said animal.

Preferably the peptide is that of the invention described earlier.

The term “milk producing animal” refers to any animal which produces milk either for human consumption or for suckling its young, and includes cows, goats, sheep, camels and the like.

In another preferred aspect the invention provides a method of decreasing the activity of the c-fos gene and/or increasing the activity of the c-jun gene in an animal, comprising the step of administering an effective amount of a peptide based on SSTR to said animal.

Preferably the peptide is that of the invention described earlier.

In another preferred aspect the invention provides a method of altering calcium metabolism in an animal, comprising the step of administering an effective amount of a peptide based on SSTR to said animal.

Preferably the peptide is that of the invention described earlier.

Without wishing to be bound by theory, it appears that administration of a peptide based on SSTR affects the activity of the c-fos and c-jun genes and therefore calcium metabolism. This results in changes in muscle function. For example animals treated with the peptide have shown improved water holding capacity in the muscle tissue. The improvement is in the order of 20%.

In a related aspect the invention provides a method of stimulating an immune response to a hormone, carrier protein, binding protein or a hormone receptor in an animal wherein said immune response modulates hormone activity, said method comprising the step of administering an immune response-inducing effective amount of the peptide of the first aspect of the invention to said animal.

In another related aspect the invention provides a method of stimulating an antibody response to a hormone, carrier protein, binding protein or hormone receptor wherein said response modulates hormone or receptor activity in an animal and wherein said antibody response results in antibodies being secreted on the mucosa of the animal and/or in the milk of said animal, said method comprising the step of administering to said animal an antibody stimulating effective amount of the peptide of the invention.

Preferably the mucosa onto which antibodies are secreted is the lung, mammary, gastrointestinal and/or urogenital mucosa.

In a further aspect, the invention provides a kit comprising the novel peptides or molecule of the invention, optionally further comprising the immuno-adjuvant oil of the invention.

### DETAILED DESCRIPTION OF THE INVENTION

The invention will now be described with reference to the following non-limiting Figures and Examples.

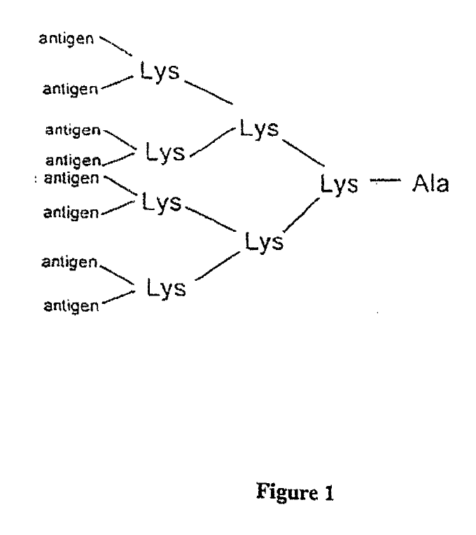

FIG. 1 is a diagrammatic representation of multiple antigen peptide (MAP) showing 7 branching lysine with peptide antigens.

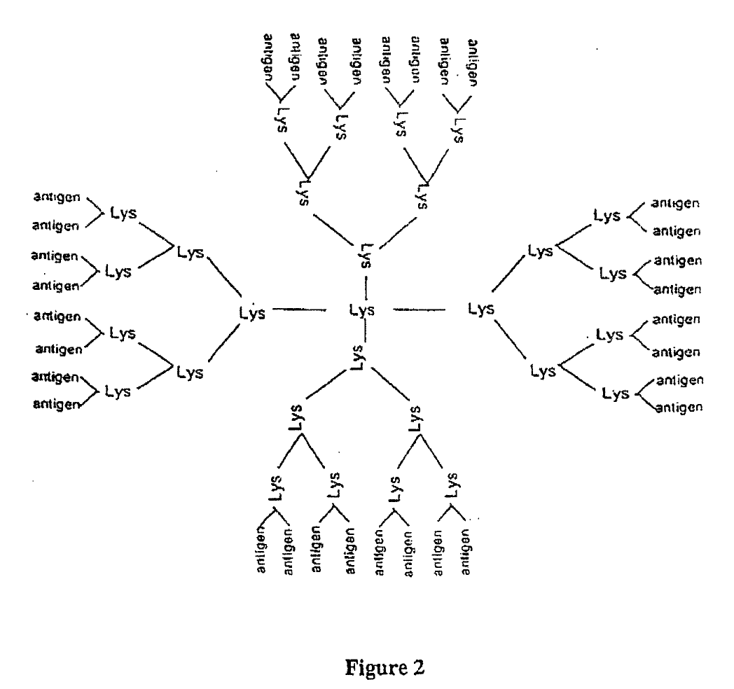

FIG. 2 is a diagrammatic representation of MAP showing 16 branching lysine with 36 peptide antigens.

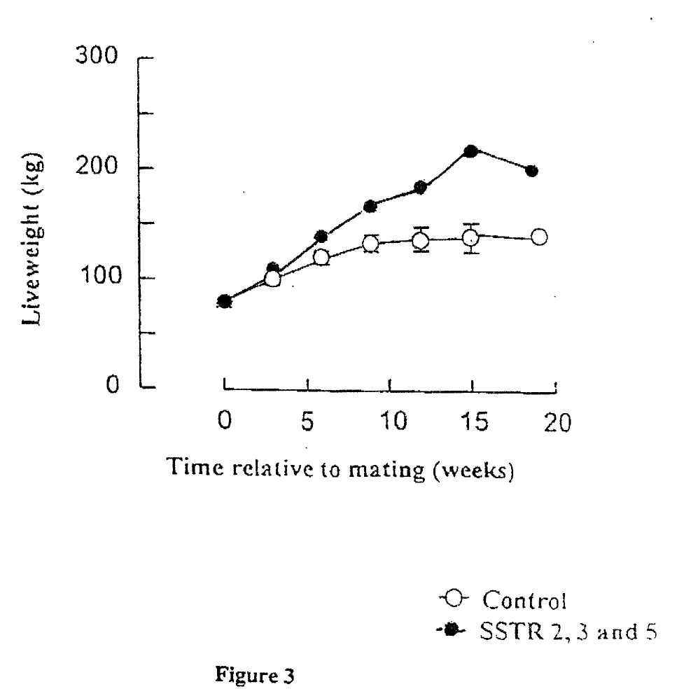

FIG. 3 is a graphic representation of live weights of pigs during pregnancy and lactation in Example 6.

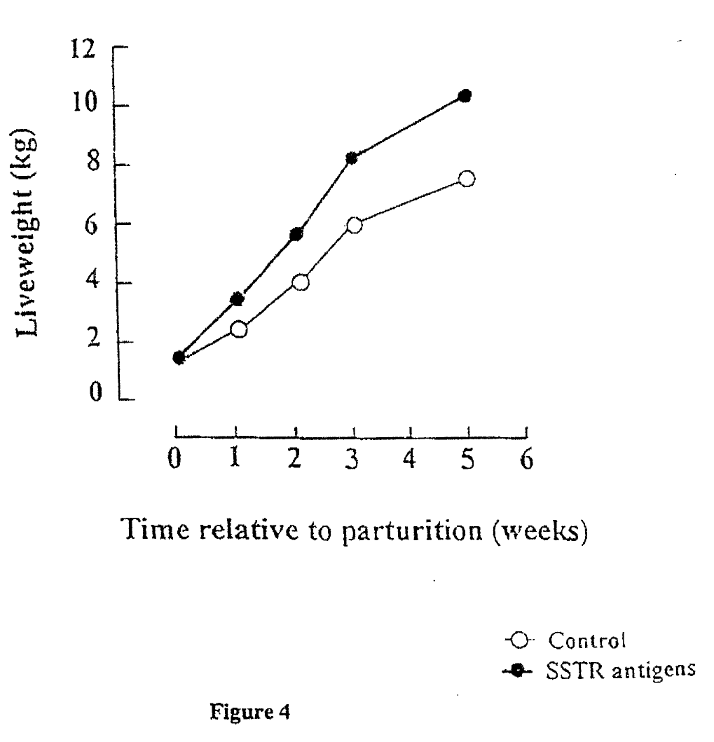

FIG. 4 is a graphic representation of live weights of piglets in Example 6.

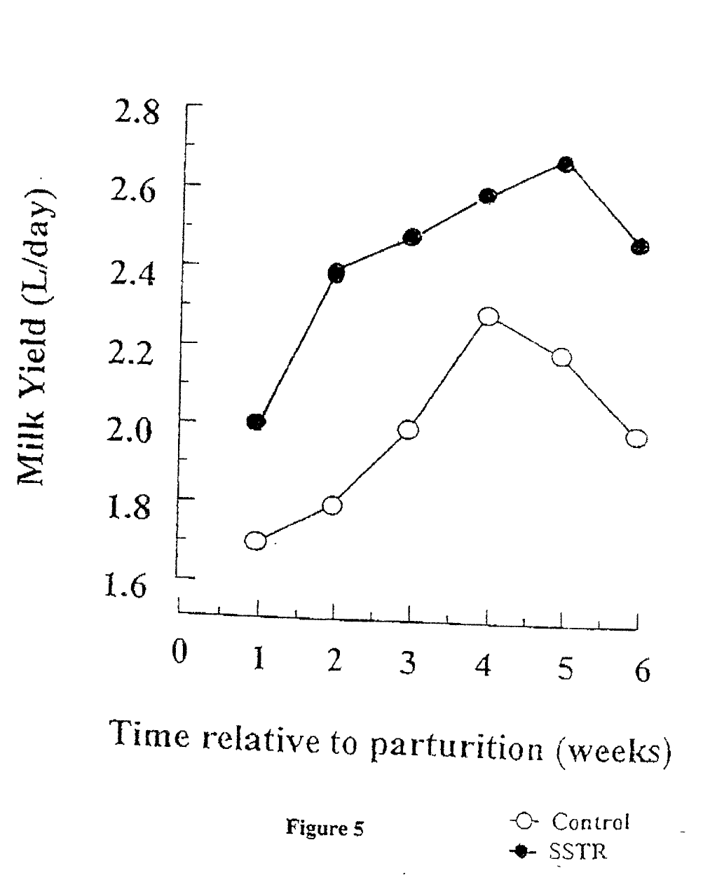

FIG. 5 is a graphic representation of milk produced by cows in Example 9.

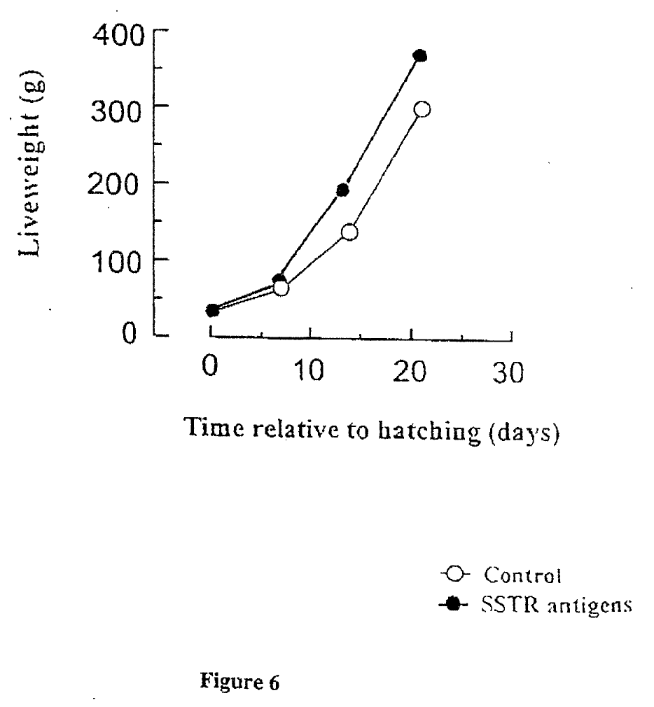

FIG. 6 is graphic representation of live weight of chickens in Example 10.

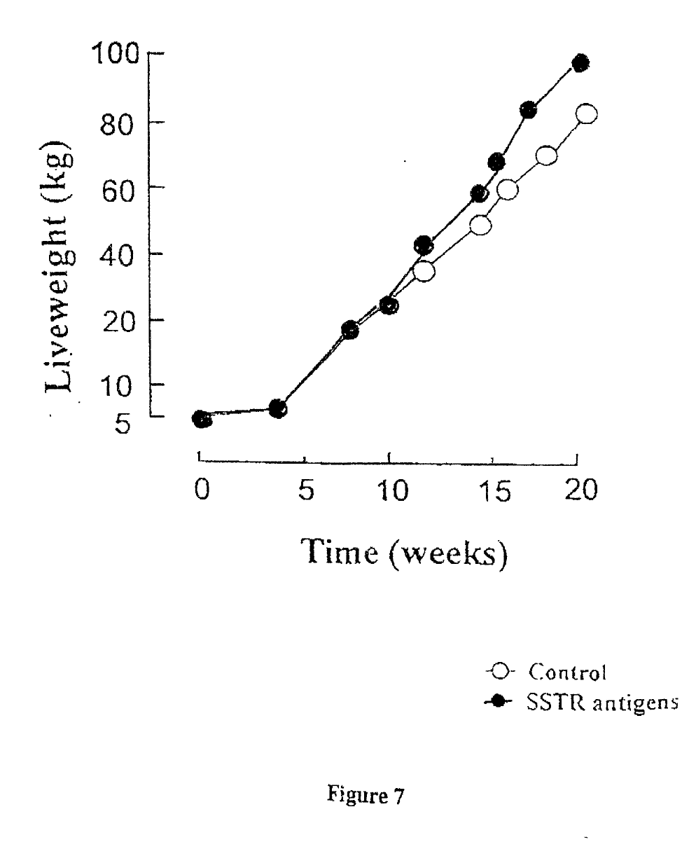

FIG. 7 is a graphic representation of live weight of pigs in Example 11.

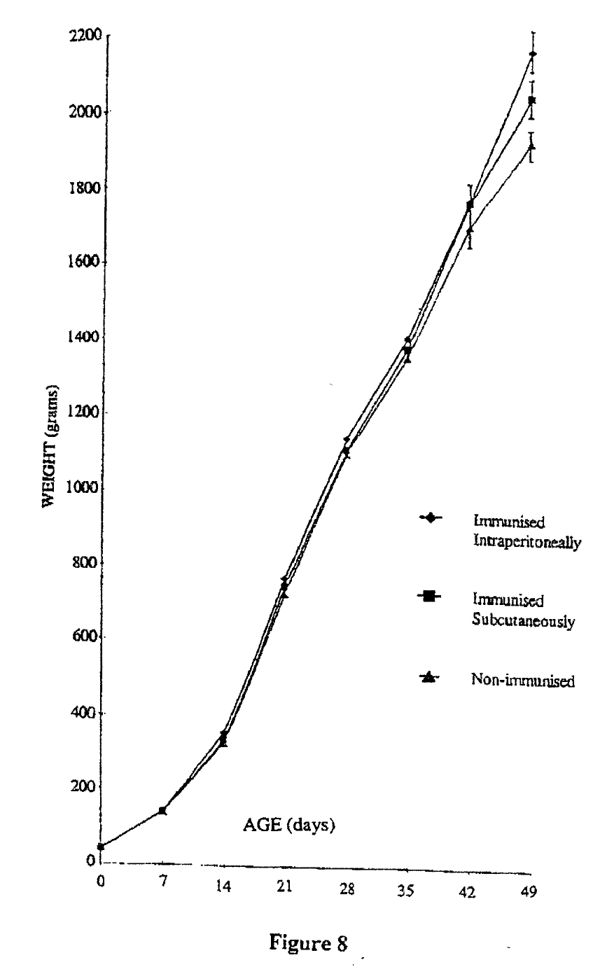

FIG. 8 is a graphic representation of live weight of chickens in Example 14.

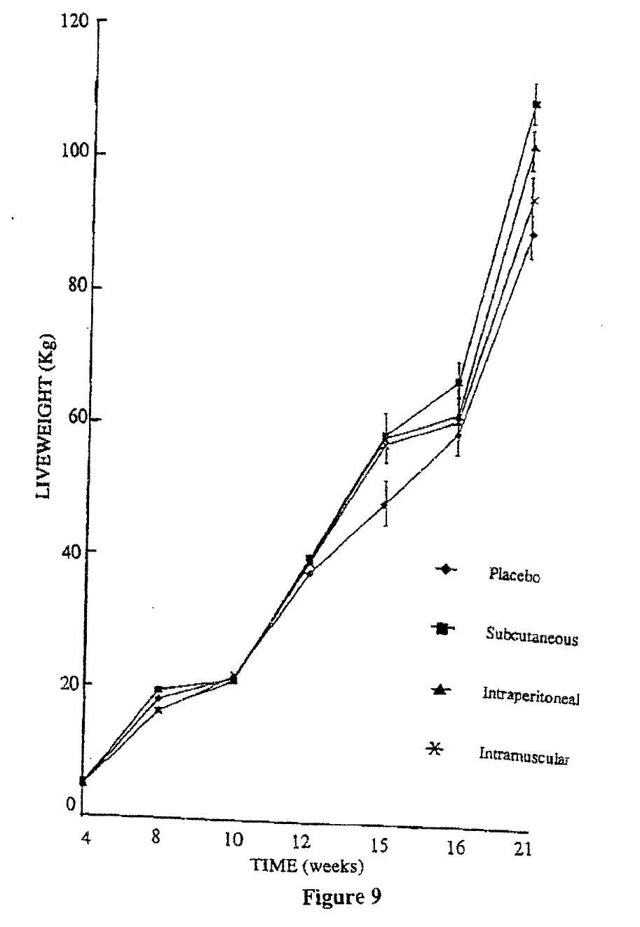

FIG. 9 is a graphic representation of live weight of male pigs in Example 15.

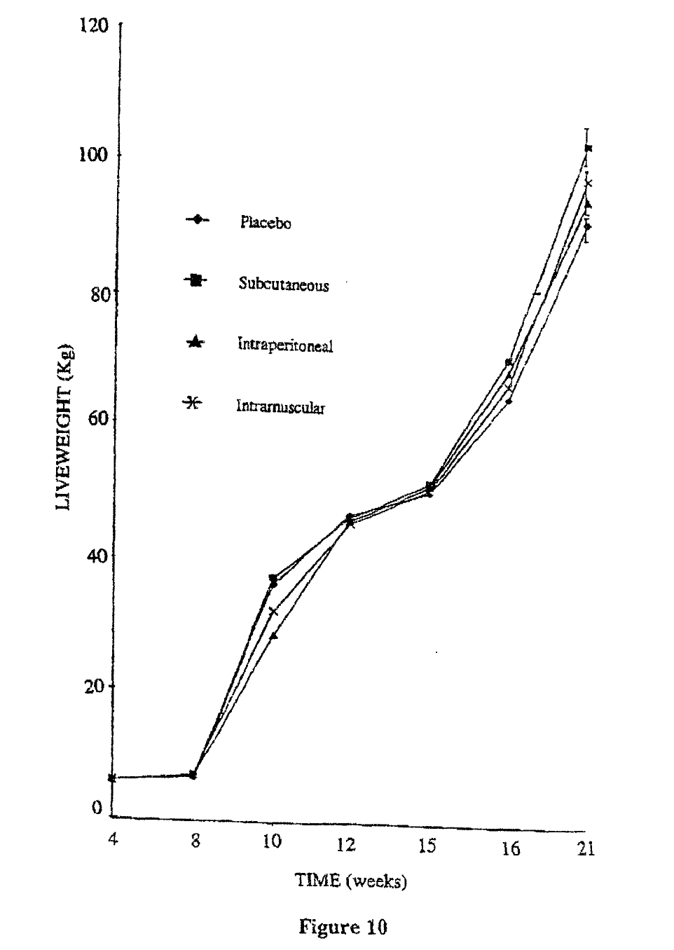

FIG. 10 is a graphic representation of live weight of female pigs in Example 15.

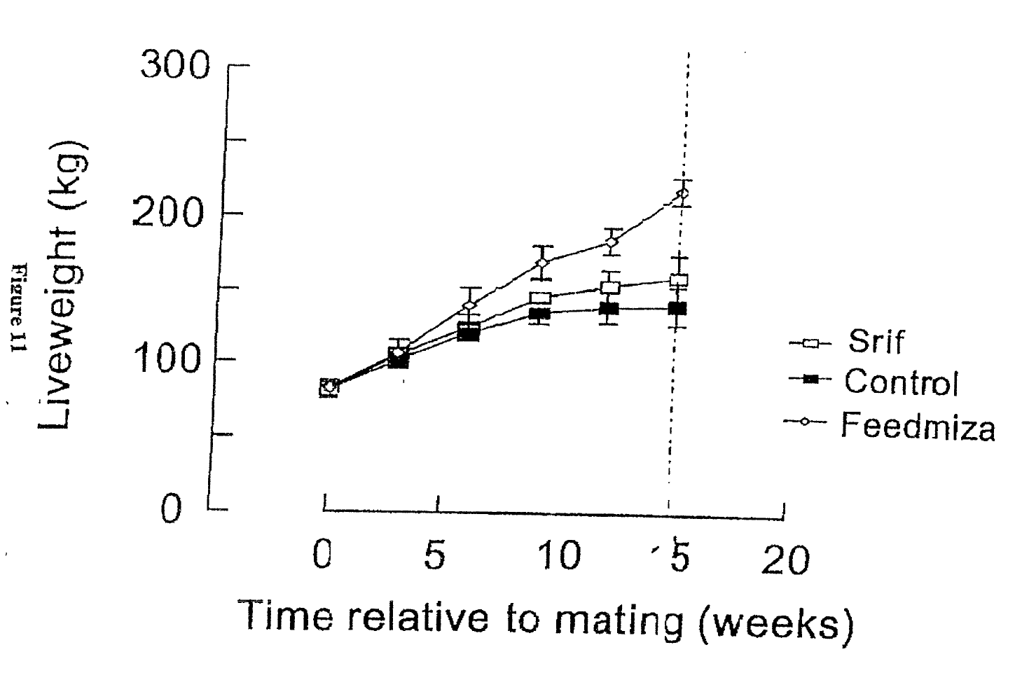

FIG. 11 is a graphic representation of the live weight of pigs in Example 16.

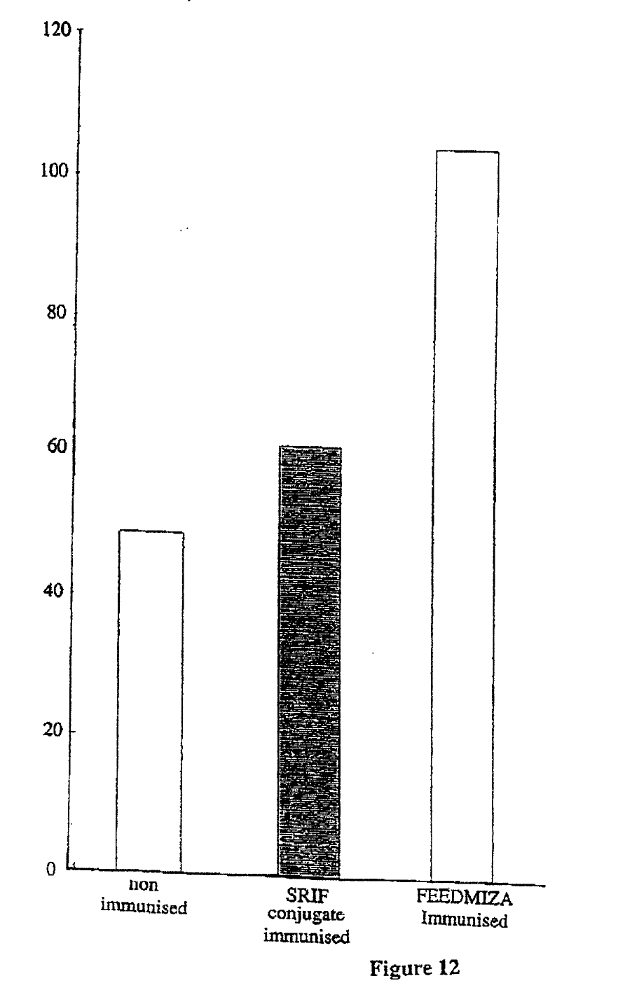

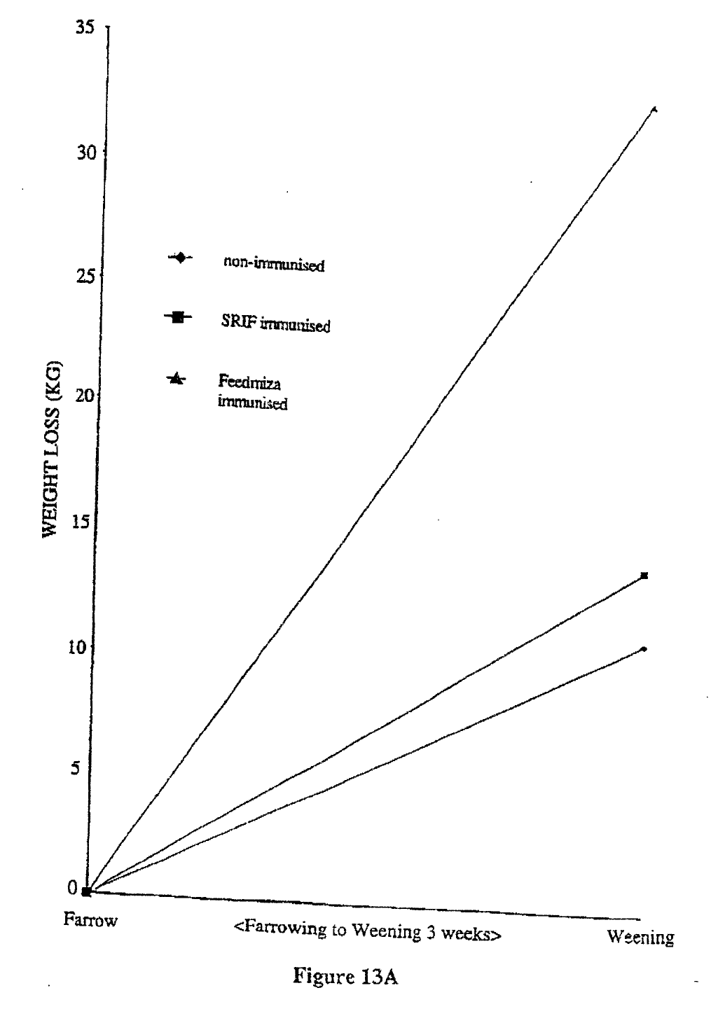

FIG. 12 is a graphic representation of the net, overall live weight gain of sows in Example 17.

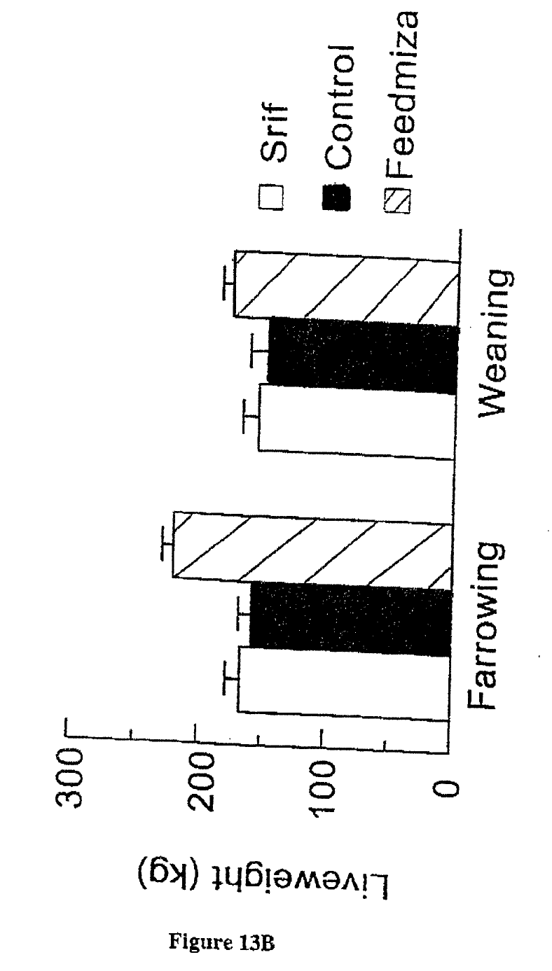

FIG. 13 is a graphic representation of the live weight of the sows in Example 17.

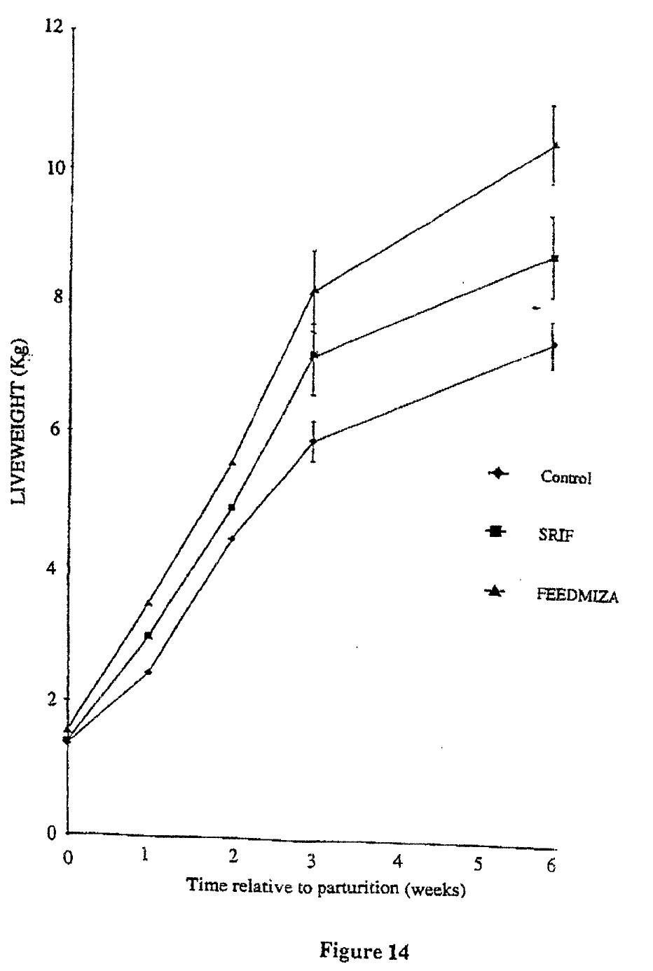

FIG. 14 is a graphic representation of the live weight of the piglets in Example 18.

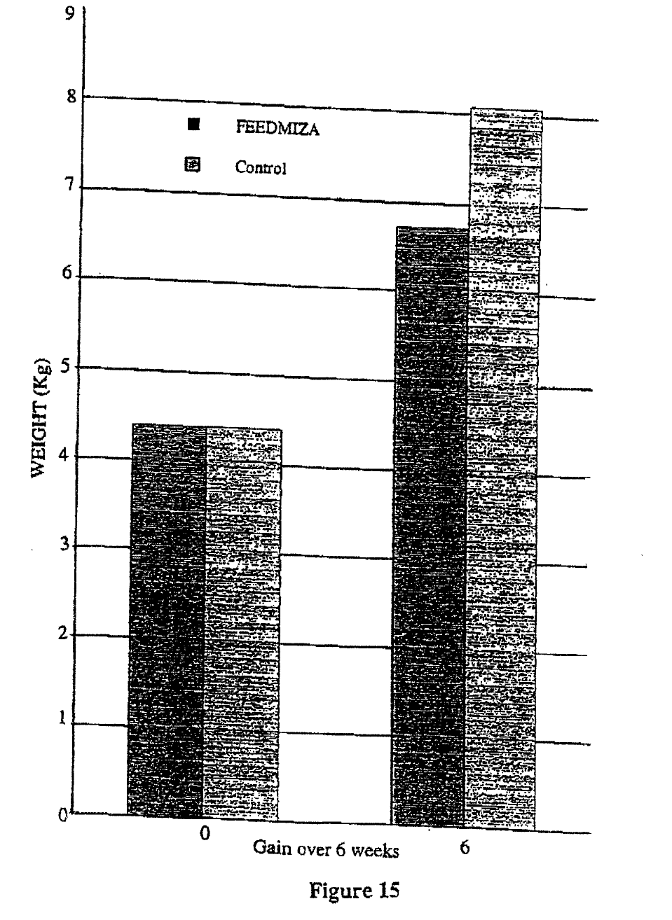

FIG. 15 is a graphic representation of the live weight of the twin lambs in Example 19.

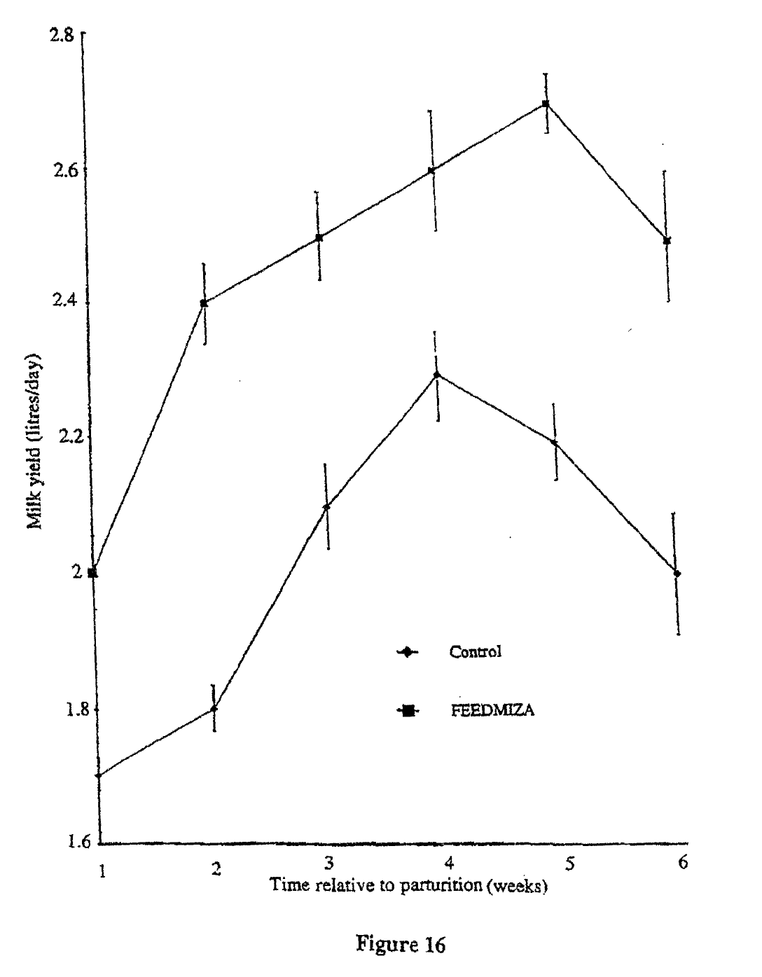

FIG. 16 is a graphic representation of the yield of milk from the ewes in Example 19.

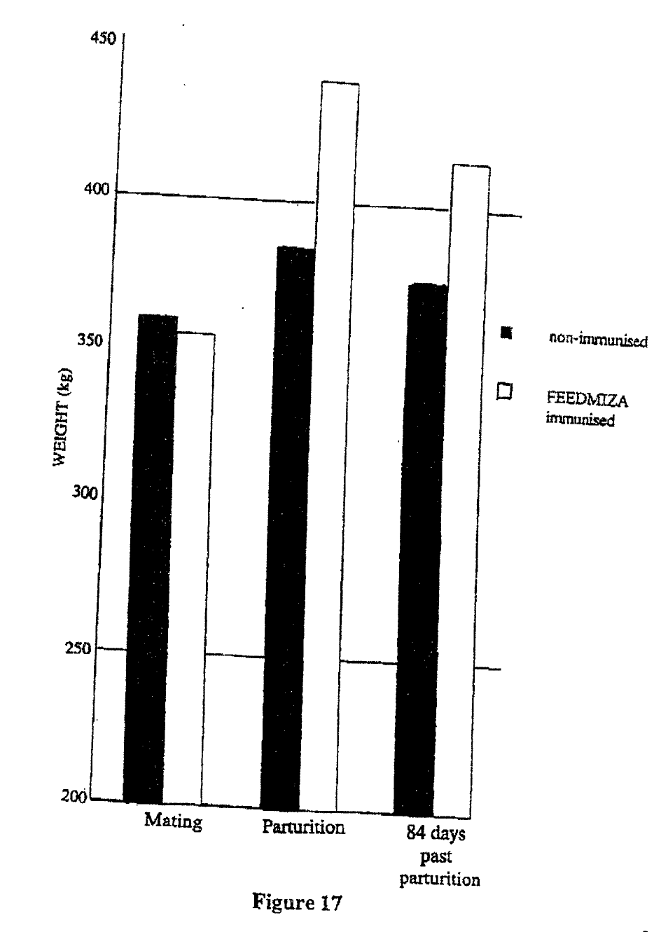

FIG. 17 is a graphic representation of the live weight of the cows in Example 20.

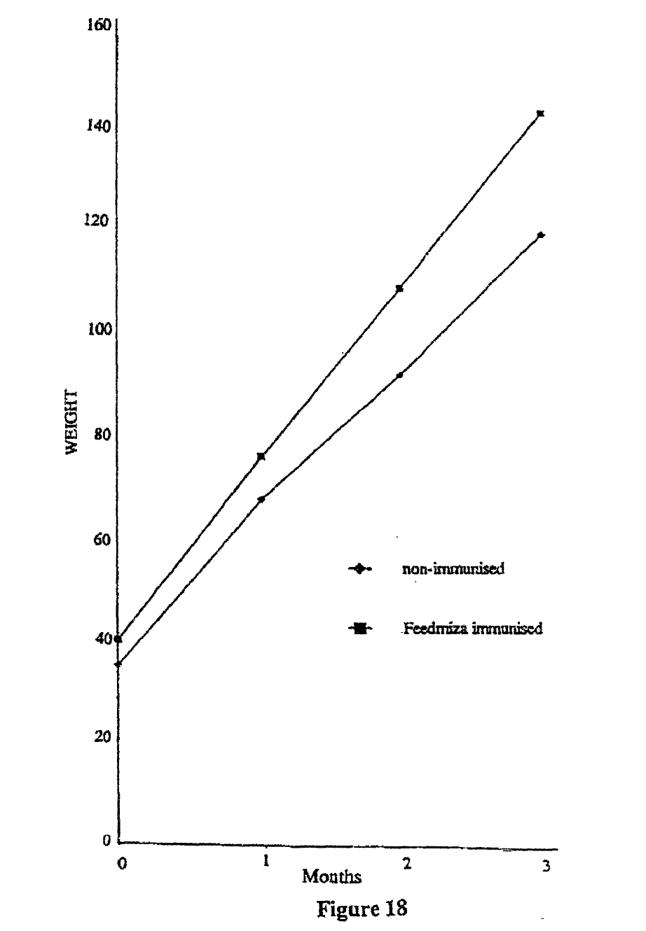

FIG. 18 is a graphic representation of the live weight of the calves in Example 20.

### EXAMPLE 1

### Preparation of the Peptides

Peptides were synthesised using a solid phase automated peptide synthesiser (for example Advanced ChemTech ACT Models 396, 348 or 90).

The purity of these peptides is determined by analytical HPLC and an amino acid analyser. The quality of the peptides of this invention was further supported by amino acid composition analysis. The peptides were then lyophilised and stored at −20° C.

### EXAMPLE 2

### Enzyme-linked Immunosorbent Assay

An enzyme-linked immunosorbent assay (ELISA) was developed to measure anti-SSTR or anti-IGFBP antibodies in biological fluids; plasma, fat-free colostrum, milk, and gastric mucosa. Multi-well plates were coated with 100 μL of phosphate buffered saline (PBS; pH 7.4) containing 5 μg mL⁻¹ of a complex of SSTR and/or IGFBP antigens and ovalbumin (Sigma Chemical Co., St Louis, U.S.A.) for 16 hours at 4° C. The antigen-coated plates were then rinsed three times with PBS containing 5% (w/v) skim milk (“Coffee Mate”; Carnation, Nestle, Sydney, Australia). To prevent the incidence of non-specific binding, the remaining absorbent sites were blocked by dispensing 100 μL of PBS plus 5% skim milk solution per well for 2 hours at room temperature. At the cessation of the “blocking” period (2 hours) and subsequent steps, the plates were washed three times with a solution of PBS containing 0.1% (v/v) Tween-20 (PBST). To each well was added an aliquot of 100 4 μL of diluted sample (1/400 v/v; plasma, fat-free colostrum or gastric mucosae in PBST) and the plates were incubated for a further 2 hours at room temperature. Next, an aliquot of 100 μL of goat anti-porcine IgG-Fc fragment or goat anti-porcine IgA-Fc fragment (1/400 (v/v) in PBST; Nordic Immunological Laboratories, Tilburg, The Netherlands) was added to each well and plates were incubated for a further 2 hours at room temperature. The final incubation for each well was with 100 μL of a 1/400 dilution of horseradish peroxidase-conjugated rabbit anti-goat IgG-H+L fragment (Nordic Immunological Laboratories, Tilburg, The Netherlands) in PBST plus 5% skim milk, for 2 hours. Prior to the addition of the substrate, the final wash consisted of two washes with PBST then one wash with distilled water. The plates were developed using 100 μL of substrate [1 mM 2,2′azino-di (3-ethylbenzthiazoline sulphonate) crystallised diammonium salt (ABTS; Sigma Chemical Co., St Louis, U.S.A.) and 2.5 mM H₂O₂ in 10 mL citrate phosphate buffer (0.1 M citric acid, pH was adjusted to 4.2 with 0.5 M Na₂HPO₄)] per well and absorbances were read after 30 minutes and 60 minutes with a Titertek MC plate reader at 450 nm. Each sample was tested in duplicate and both positive and negative controls were included for each ELISA plate.

Titres of anti-SSTR or IGFBP antibodies in the plasma, colostrum, milk and gut mucosa were expressed as the ratio of the optical density (OD) reading for positive control sample relative to the OD reading for the test sera (Steward and Lew, 1985; Reynolds et al., 1990). This ratio was multiplied by the dilution factor of each sample to establish relative quantities of each isotype produced.

Antibody titre=(OD₄₅₀ test sera)×(OD₄₅₀ standard)⁻¹×sample dilution⁻¹

The avidities of anti-SSTR or IGFBP antibodies were determined using scatchard plot analyses as outlined by Holst et al., (1992a).

### EXAMPLE 3

### Preparation of the Peptide Complex

It is preferred that a multiple antigen peptide (MAP) system is employed to present/carry the antigens to the immune system. This approach to prepare peptide immunogens of relatively small molecular weight overcomes the ambiguity of conventional carrier systems (i.e. KLH, BSA thyriodglobulin etc.). The MAP approach produces chemically defined peptide antigens with a high degree of homogeneity. As a consequence immunogens prepared by the MAP approach elicit high and uniform antibody response to the immunogen in immunised animals. As such, the MAP approach to presenting the antigens described in the present application is particularly suitable for eliciting a uniform and site-specific antibody response to the desired antigens.

Historically the MAP system consists of an oligomeric branching lysine core, usually composed of three or seven lysines, and four or eight copies of dendritic arms of peptide antigens (see FIG. 1). However, the MAP system employed in the present application consists preferably of 18 to 20 branching lysine core giving rise to 36 to 40 copies of dedritic arms of the peptide antigens (see FIG. 2). Since each peptide arm may consist of 5-20 amino acids, the overall appearance of the MAP system is of a macromolecule with a high density of surface peptide antigens and a molecule weight exceeding 40,000.

The MAP system can be used to present a single antigen alone or any combination of antigens thereof.

Furthermore, the antigen/carrier system displays no apparent biological activity, even if injected actively.

The MAP system described in the present application was synthesised using a solid phase automated peptide synthesiser (for example Advanced ChemTech ACT Models 396, 348 or 90).

The MAP/antigen is then suspended (ideally emulsified) into a non-inflammatory delivery vehicle or immunostimulator (coded as NSB-050). In this form the product is administered usually by intraperitoneal or subcutaneous injection. The product may also be injected intramammarily, intramuscularly, or delivered orally. Following subcutaneous or intraperitoneal injection the emulsion rapidly is taken up by the lymphatic system, and presented to the immune system. At this point there is attachment of the various antigens to receptor sites on the processing cell of the immune system, resulting in the production of the specific antibodies of high titre and affinity.

### EXAMPLE 4

### The Delivery Vehicle

A clear oily liquid has been identified as particularly useful in developing antibodies which are secreted on the epithelial surfaces of the lung, gastrointestinal and urogenital tracts, the mucosa of the mammary and thus subsequently the colostrum and milk of animals immunised actively. Furthermore, the delivery system (NSB-050) is a particularly effective immunostimulator when attempting to elicit an immune response to a number of different antigens concurrently.

In general, the oil has the following

| composition: | |
|---|---|
| diacylglycerol ethers | 30-50% |
| triacylglycerols | 40-70% |
| polar lipids | 10-15% |
| free fatty acids | >2% |
| hydrocarbons | >2% |
| wax esters | >2% |

Specifically, the oil is of the following typical analysis:

octadec-9-enylglyceryl ether 45%(20-70)

1-hexadecylglyceryl ether 11%(3-25)

hexadec-7-enylglyceryl ether 5%(1-15)

octadecyl glyceryl ether 7%(1.5-20)

eicosa-9-enylglyceryl ether 5%(1-15)

lecithin 13% (1-25)

DL alpha tocopherol acetate 7% (1-25)

1,2,5-dihydroxycholecalciferol 1%(0-3)

Vitamin A 1%(0-5)

Non-mineral oil (vegetable or animal origin)5%(0-40)

The delivery vehicle, except for lecithin and non-mineral oil (vegetable or animal origin) are oils extracted from the livers of deep sea sharks, specifically the livers of the Pacific sleeper shark (Somniosus pacificus) and the Plunket shark (Centroscymnus plunketi). The oils are recovered from the livers of these sharks at less than or equivalent to 125° C. and at a pressure less than or equivalent to 666.6 Pa.

Ideally, the livers are collected fresh from these sharks which are preferred because the oils are devoid of or contain minimum levels of hydrocarbons and wax esters. The excised livers are washed in fresh sea or tap water, and then macerated. The macerated mass is allowed to stand at room temperature (ideally 25° C.) for 3 hours, after which the oil is decanted. Clarification of the oil occurs firstly by centrifugation, then by washing with water, followed by a deproteinisation step with bentonite, or similar material. Following a further washing of the oil, it is then stored at 4° C. storage for a few weeks to sediment out any winterable material. The clear supernatant is then decanted, mixed with the lecithin emulsifier, and with any of the oil soluble vitamins desired to form the oily delivery vehicle. It is stored in appropriate containers and treated with nitrogen gas to prevent oxidation.

### EXAMPLE 5

### Preparation of the Vaccine

The constructed protein molecule was dispersed in phosphate buffered saline (pH 7.4) by use of ultrasonic agitation. (It is helpful to the performance of the immunogen if the antigen is not soluble in the aqueous phase, and if the protein is dispersed into single molecules in the liquid). The antigen was mixed preferably in saline (aqueous phase) and emulsified with the oil phase at the rate of 2 parts of oil to 1 aqueous part to produce a stable water-in-oil emulsion. Ideally the concentration of antigen in the aqueous phase is such that a 3 ml dose of emulsion contains 100 μg of antigen macromolecule. Generally the amount of oil in the dose is 2 ml (about 66.6% of the total volume of the vaccine), the remaining 1 ml accounts for the aqueous phase containing phosphate buffered saline and antigen.

### EXAMPLE 6

### Administration of Vaccine

The vaccine may be injected by the intraperitoneal, or subcutaneous routes (for preference), but may also be administered via intramammary, intramuscular, or oral routes. Booster injections (preferable two) were generally administered within given 2 weeks or longer after the primary vaccination.

### EXAMPLE 7

### Administration of Peptides SEQ ID NO:12, 14, 21, 22, 36, 38, 42 & 44 to Pigs

Gilts

Thirty primiparous pigs (Landrace×Large White) were mated naturally to cross-bred boars. Approximately 10 days prior to parturition, gilts were transferred to farrowing crates in an enclosed shed maintained at approximately 24° C. and lit artificially by fluorescent lighting with a 12 hour dark/light cycle.

Piglets

In an attempt to minimise the effect of litter size on milk yield and the subsequent growth of sucking piglets (King et al., 1993), the number of piglets per litter was standardised to eight.

Experimental Procedures

Prior to mating the gilts were divided randomly into six groups of five such that those gilts in group 1 received placebo injections and pigs in groups 2, 3, 4, 5 and 6 were immunised against SSTR 1 (peptide #12 and 14), SSTR 2 (peptide #21 and 22), SSTR 3 (peptide #26 and 38), SSTR 4 (peptide #42 and 44) and SSTR 5 (peptide #48 and 50) respectively. During pregnancy both immunised and non-immunised gilts were injected subcutaneously in the neck with 3 ml of the corresponding vaccine (SSTR 1 (peptide #12 and 14), 2 (peptide #21 and 22), 3 (peptide #36 and 38), 4 (peptide #42 and 44) and 5 (peptide #48 and 50) coupled to the MAP and emulsified in NSB-050) at c. 40, 65 and 90 days after mating.

Liveweights of the gilts/sows were measured each week from mating to parturition, thence at weaning which occurred at three weeks post partum. Immediately after parturition, birthweights of piglets were recorded and thereafter liveweights were measured at weekly intervals until weaning, thence at five weeks of age.

On day three post partum eight piglets passively immunised via the dam and a further eight control piglets were modified surgically to determine gastric acidity, portal blood flow, plasma hormones and MCA in response to intravenous infusions of pentagastrin at the rate of 10 μg kg liveweight⁻¹ hour. A further eight piglets from each group were treated similarly on day 21 post partum.

Results

Effects of Immunisation on Sows

There were no obvious lesions at the sites of injection or any detrimental side effects which may have been attributed to the vaccination regime imposed for immunised or control sows.

Liveweights

Sows

As depicted in FIG. 3 those gilts immunised against SSTR 2 (peptide #21 and 22), SSTR 3 (peptide #36 and 38), and SSTR 5 (peptide #48 and 50) antigens were significantly heavier at parturition and tended to lose more weight during the subsequent lactation than corresponding control sows.

Piglets

The total numbers of piglets (dead or alive) and the number of males and females born per litter did not differ significantly for immunised or control sows.

Piglets from sows immunised against SSTR 2 (peptide #21 and 22), SSTR 3 (peptide #36 and 38), and SSTR 5 (peptide #48 and 50) were significantly heavier at birth relative to those piglets from control sows or sows immunised with SSTR 1 and 4 (see Table 2).

Data for the growth of piglets are summarised in Table 2 and FIG. 4. After three weeks from birth, piglets from sows immunised with SSTR 1 (peptide #12 and 14), SSTR 2 (peptide #21 and 22), SSTR 3 (peptide #36 and 38), SSTR 4 (peptide #42 and 44) and SSTR 5 (peptide #48 and 50) during pregnancy grew significantly faster than corresponding piglets from non-immunised dams. This difference in growth rates of piglets from immunised sows was maintained throughout the experiment such that, at five weeks of age, piglets from immunised dams were some 20-30% heavier than those piglets from corresponding control sows.

The above differences were apparent for piglets of both sexes throughout the period of the experiment. Thus, male and female piglets of immunised sows grew significantly faster (P<0.01) than corresponding piglets from control sows.

Antibodies in Blood, Colostrum and Gut Scrapings

Mean titres of antibodies to SSTR 1 (peptide #12 and 14), SSTR 2 (peptide #21 and 22), SSTR 3 (peptide #36 and 38), SSTR 4 (peptide #42 and 44) and SSTR 5 (peptide #48 and 50) in the colostrum of immunised and control sows are shown in Table 3 Titres of anti-SSTR antibodies in the plasma and gut scrapings of piglets, collected prior to and after the gastric infusions studies on days three and 21 are summarised in Table 3. Throughout the study, antibodies to SSTR were not detected in the colostrum of non-immunised sows or in the plasma and stomach mucosa of piglets sucking control dams. In contrast, high titres of SSTR antibodies were detected in the colostrum of all immunised sows, as well as in the plasma and mucosal scrapings of piglets sucking immunised dams at both three and 21 days post partum.

Titres of anti-SSTR IgG antibodies were significantly greater (P<0.01) than levels measured for anti-SSTR IgA antibodies in the colostrum collected from immunised sows at, or near, the time of parturition. Similar differences in the levels of IgG relative to IgA anti-SSTR antibodies were recorded in the mucosa scrapings of piglets sucking immunised dams; this was more apparent for piglets at 21 than at three days of age. In contrast, no significant differences were measured for titres of IgG and IgA anti-SSTR antibodies in the plasma of immunised piglets on days three and 21. Furthermore, levels of IgG antibodies measured in the stomach mucosa did not differ significantly (P>0.10) from levels detected in plasma of immunised piglets, at both three and 21 days post partum. However at all times, titres of anti-SSTR IgA antibodies in the plasma were significantly (P<0.05) greater than levels detected in the gastric mucosa for those piglets from immunised sows.

Feed Intakes of Sows

Throughout the period from parturition to weaning, immunised and control sows consumed all offered feed. Thus, the efficiency of utilisation of food for growth of sucking piglets and/or for milk production was significantly greater (P<0.05) for immunised than control sows.

Hormones and Gastric Function

Plasma samples collected from piglets over the three week period until weaning showed that there was no significant difference in the concentrations of growth hormone, thyroid hormones or glucagon for piglets sucking immunised or control sows. However, levels of circulating concentrations of insulin and IGF I and II were significantly greater for immunised piglets relative to corresponding control piglets.

The level or amount of gastric acid secretion following pentagastrin stimulation was observed to be retarded significantly for those piglets sucking immunised than control dams. Furthermore, the activity of key gastric enzymes were suppressed in these piglets. The results from these studies suggest that antibodies which block the SSTR 1 through to 5 alter gut gut function and thereby improve digestion.

### EXAMPLE 8

### Administration of Peptides SEQ ID NO: 18, 20, 36, 38, 48 & 50 to Sheep

Twenty pregnant merino sheep were selected randomly at 40 days of pregnancy and divided into two groups of ten; immunised and control. During pregnancy immunised ewes were administered subcutaneously in the neck a 3 ml emulsion of the antigen in NSB-050 at c. 90, 110 and 132 days of pregnancy. Placebo injections were administered to control ewes at the corresponding times. The antigens used for immunisation comprised of the SSTR 2 (peptide #18 and 20), SSTR 3 (peptide #36 and 38) and SSTR 5 (peptide #48 and 50) coupled individually to the MAP system and were administered concomitantly.

At weaning (3 months post partum) there was no significant difference in liveweights for immunised relative to corresponding control ewes. Liveweights of those lambs sucking immunised dams were some 20% heavier than those sucking control ewes.

The yields of wool were approximately 10% greater for immunised than control ewes during pregnancy and subsequent lactation. Furthermore, the yields of wool for lambs consuming colostrum/milk from immunised dams was improved by some 20% than corresponding control lambs. In addition the wool harvested from immunised lambs was significantly finer than that collected from control lambs. The changes in wool characteristic has been postulated to be attributed to improved nutritional status of those lambs sucking immunised dams leading to greater populations of secondary and primary follicles (see Table 4).

### EXAMPLE 9

### Administration of Peptides SEQ ID NO:18 20, 36, 38, 48 & 50 to Cattle

Twenty pregnant beef heifers were selected from a grazing herd and allocated to two groups of ten; immunised and control. Immunised cows were injected subcutaneously in the neck with a complex of SSTR 2 (peptide #18 and 20), SSTR 3 (peptide #32 and 34) and SSTR 5 (peptide #48 and 50) antigens coupled to the MAP system emulsified in NSB-050 at c. 5, 7 and 8 months of pregnancy. The remaining ten cows were injected with placebo injections administered subcutaneously in the neck at the corresponding times. Calves from immunised cows were significantly heavier at birth (38 v 46 kg; P<0.05) than corresponding calves from non-immunised cows. During a ten week period calves sucking dams immunised against SSTR 2 (peptide #18 and 20), SSTR 3 (peptide #36 and 38) and SSTR 5 (peptide #48 and 50) grew significantly faster (by some 10-15%) than calves from control cows.

### EXAMPLE 10

### Administration of Peptides SEQ ID NO: 18, 20, 36, 38, 48 & 50 to Sheep

Twenty-four crossbred ewes (Merino×Dorset Horn) were allocated to two treatment groups; immunised (n=12) and control (n=12). Immunised ewes were injected subcutaneously in the neck with a complex of SSTR 2 (peptide #18 and 20), SSTR 3 (peptide #36 and 38) and SSTR 5 (peptide #48 and 50) peptides and MAP emulsified in NB 050, the remaining ewes received placebo injections. Ewes were administered corresponding injections at approximately 90, 110 and 130 days of pregnancy. Over a six week period from the time of parturition immunised ewes produced significantly more milk (20%) than non-immunised ewes (see FIG. 5). The increase in milk yield observed for immunised ewes could not be accounted for by increased appetite, thus immunised ewes utilised their feed more efficiently than control ewes.

### EXAMPLE 11

### Administration of Peptides SEQ ID NO: 27, 29, 36, 38, 48 & 50 to Chickens

Forty day old chickens were allocated randomly two treatment groups; immunised and control. Immunised birds were injected intraperitoneally at one day age and subsequent booster vaccinations were administered orally at 7 and 14 days of age with 20 μg of a complex of SSTR 2 (peptide #27 and 29), SSTR 3 (peptide #36 and 38) and SSTR 5 (peptide #48 and 50) coupled to the MAP system emulsified in NSB 050. Control birds received placebo injections at the corresponding site and time.

Over the first 4 weeks after the initial vaccination immunised birds were significantly heavier than corresponding control birds such that at the cessation of the experiment immunised birds were some 24% heavier than control birds (see FIG. 6).

At 21 and 42 days of age a representative proportion (n=5) of birds from each group were selected randomly and euthanised for muscle analysis The patagialis muscle was dissected from the each of the birds. The yield of wet muscle collected from immunised birds was recorded to be some 23% and 30% heavier than control birds at 21 and 42 days of age respectively. Although the mass of the patagialis muscle for immunised birds was observed to be heavier than corresponding control birds, the total yield of RNA per gram of muscle tissue was significantly less for immunised relative to control birds. When identical amounts of recovered DNA from the patagialis muscle of both groups of birds were titrated for the c-fos and c-jun genes it was observed that the copy numbers of the c-fos gene was suppressed significantly and c-jun gene was increased relative to control birds.

It is apparent from the results of the present study that vaccination against SSTR 2 (peptide #27 and 29), SSTR 3 (peptide #36 and 38) and SSTR 5 (peptide #48 and 50) increased liveweight gain of immunised birds. Furthermore, it is evident that intracellular mechanisms of key muscle systems were altered significantly following immunisation. The immunisation procedures resulted in the suppression of the transcriptional regulator gene c-fos and amplified activity of c-jun. The changes in the activity of these genes observed for immunised relative to control birds is hypothesised to be associated with altered transcription of protein to protein binding relationships resulting in changes in the signal transduction pathways and thus increased calcium metabolism.

### EXAMPLE 12

### Administration of Peptides SEQ ID NO: 21, 23, 36 & 38 to Pigs

Twenty six week old cross-bred male pigs were allocated randomly to two treatment groups; immunised and control. Pigs were injected subcutaneously in the neck with either a mixture of SSTR 2 (peptide #21 and 23), SSTR 3 (peptide #36 and 38) and SSTR 5 (peptide #48 and 50) antigens coupled to the MAP system emulsified in NSB 050 or placebo injections at 42, 63 and 84 days of age. At the cessation of the experiment (20 weeks of age) pigs immunised against SSTR were significantly heavier (91.5 v 83.5 kg) than corresponding control pigs (see FIG. 7).

### EXAMPLE 13

### Administration of Peptides SEQ ID NO: 54 to 57 to Rats

Twenty laboratory rats were allocated randomly to two treatment groups immunised and control. Immunised rats were injected subcutaneously in the medial thigh with a 3 ml emulsion containing 100 μg of a mixture of IGFBP 1 (peptide #47), IGFBP 2 (peptide #55), IGFBP 3 (peptide #56) and IGFBP 4 (peptide #57) antigens described in the present patent coupled to the MAP system suspended in NSB 050. Control rats received placebo injections administered subcutaneously in the medial thigh. The rats were administered three booster injections at intervals of 21 days with the corresponding vaccines. Blood samples were collected over the period of the study for antibody titres and concentrations of key metabolites and hormones. Over the study those rats immunised with the IGFBP antigens grew significantly faster and the concentrations of glucose (2.5 v 4 mM) and insulin (20 v 30 ng/ml) were suppressed relative to corresponding control rats. The above vaccination regime may have profound effects on glucose metabolism in diabetes syndromes Type I and II.

### EXAMPLE 14

### Administration of the Peptide SEQ ID NO:1 (LCFWKTC to Chickens

The peptide and delivery vehicle is hereinafter referred to as FEEDMIZA

Protocol

Freshly hatched 1 day old chicks were allocated randomly into the following groups.

| | |
|---|---|
| Group A | non-immunised (n = 30) |
| Group B | immunised subcutaneously (n = 30) |
| Group C | immunised intraperitoneally (n = 30) |

Immunisations took place on the day of hatching and at 21 days of age. The FEEDMIZA dosage was 80μg of peptide in 0.1 ml of emulsion.

At 49 days, animals in each experimental group were sacrificed. In addition to the collection of blood bile and gut scrapings were collected for subsequent antibody assay.

Results

The heaviest mean liveweights of the chickens at the end of the experiments were recorded. For Group C, the weights were 2.19±0.057 kg. Mean liveweight was 2.07±0.049 kg and 1.95±0.047 kg for Group B and A respectively. The differences between each group were stastistically significant (P<0.05). The chickens which were immunised by the intraperitoneal route were approximately 12.3% heavier than the non-immunised birds. Those immunised by the subcutaneous route were an average of 7.2% heavier than the non-immunised birds. Antibodies to the FEEDMIZA antigen were detected in 100% and 60% of the samples of bile obtained from Group C and Group B respectively. No antibodies to the antigen could be detected in the bile of the birds in Group A. Antibodies were detected in gut scrapings of 100% of the Group C birds, 50% of the Group B birds, and in none of the Group A birds.

Antibodies were detected in the blood of 100% of the birds in Group B, in 40% of the birds in Group C, and in none of the birds in Group A.

Conclusions and Discussion

A significant increase in liveweight at slaughter was observed following immunisation with the FEEDMIZA preparation, with the heaviest group being those which had received the vaccination via the intraperitoneal route. It is potentially of significance that all of the chickens tested from the group which was immunised intraperitoneally showed evidence of antibody in the bile and gut scrapings, but only 40% of them showed antibodies in the blood.

In contrast the chickens from Group B which had gained significantly more weight than the non-immunised birds, but significantly less than the birds which had been immunised by intraperitoneal injection, all showed antibodies in the blood, but only half of them showed antibodies in the bile and gut scrapings.

This suggests that the birds which had been immunised by the intraperitoneal route had a immune response which differed from those birds which had been vaccinated by the subcutaneous route. The intraperitoneal immunisation had stimulated outpouring of antibody via the bile (secreted antibody) onto the surface of the intestines. It is noted that there are many receptors for the hormones targeted by the FEEDMIZA in the mucosal surface of the intestine, and that these receptors might be more amenable to, or available to antibody blocking than antibodies circulating in the blood.

This experiment has highlighted that immunomodulation which relies upon the measurement of high levels of antibody to specific hormones in the blood may be laying an emphasis on the wrong measurement. If only blood samples had been collected in this experiment to monitor immune stimulation, it would be arguable that there had been no immune stimulation in the intraperitoneal-immunised birds because no circulating antibodies could be measured. However, the presence of the antibodies in bile and gut scrapings indicated otherwise. It is noted that the birds in the group with 100% positive incidence of antibodies had the highest liveweight at slaughter, and the group with approximately 50% of the birds showing antibodies in the bile or gut scrapings performed midway between the two groups, while the non-immunised group with no gut or bile antibodies showed the lowest weights.

### EXAMPLE 15

### Administration of Peptide SEQ ID NO:1 to Pigs

Protocol

The piglets from 12 litters were weaned at 4 weeks of age and randomised into 4 treatment groups, each of which contained 12 male and 12 female piglets. Each piglet was individually eartagged and weighed. The treatment groups were:

| | |
|---|---|
| Group A | Control group- placebo injections of |
| | delivery vehicle |
| Group B | Immunised by the subcutaneous route |
| Group C | Immunised by the intraperitoneal route |
| Group D | Immunised by the intramuscular route |

The antigen used in the following experiments was the identical antigen to that used in Example 14. The micro-protein was constructed in an organic chemistry laboratory. It was maintained at −20° C. until it was dissolved in phosphate buffered saline at pH=7 (PBS) and then emulsified into the oily adjuvant just prior to use on the pigs.

All doses of vaccine used in the experiments was 3 ml of the emulsion which contained 80 μg of antigen. This dose was chosen arbitrarily, and was persisted with because success had been obtained at that rate.

The immunised groups received FEEDMIZA containing 80 μg of antigen. The placebo group received 3 ml of emulsion devoid of the antigen. Pigs were injected at 5, 8 and 12 weeks of age.

The pigs were reared in conventional weaning, growing and finishing pens. They were offered weaner, grower and finisher feeds ad libitum.

The experiment ran until the pigs reached 161 days of age (21 weeks). Pigs were individually weighed at 4, 8, 10, 12, 15, 16 and 21 weeks of age.

Results

The liveweights at 21 weeks of age, and the average daily liveweight gains of the various treatment groups are presented in Table 5 (the males) and Table 6 (the females).

FIGS. 9 and 10 show the weights of the male and female pigs respectively at different ages during the experiment.

Discussion

The heavier weights of the pigs at 21 weeks of age which had been immunised by the subcutaneous route compared with the placebo injected pigs (21.69% for the male pigs, and 13.04% for the female pigs) was stastically significant. From FIGS. 9 and 10, it is evident that there was no detectable trend in improved daily liveweight gains for the male pigs until around 12 weeks of age. At 6 weeks of age the pigs immunised by the intramuscular and intraperitoneal routes were significantly lighter than the subcutaneously immunised or placebo injected groups. However, by 10 weeks of age there were no significant differences between the weights of any of the groups.

In the case of the female pigs a similar trend was observed, except that the placebo pigs were significantly heavier than all other treatment groups at 6 weeks of age (2 weeks after the first immunisation/injection). At 10 weeks of age the group which had been immunised subcutaneously were, like their male counterparts of similar weight to the placebo group, and significantly heavier than the group immunised intramuscularly and intraperitoneally. At 12 weeks of age all of the female treatment groups were approximately of similar weights, a phenomenon which had occurred apparently some weeks earlier with the male pigs.

It is concluded that immunisation of pigs with FEEDMIZA vaccine, especially via the subcutaneous and intraperitoneal routes has produced stastically significant responses in both male and female pigs, with the benefit being measured primarily over the last 6-8 weeks period of the test.

### EXAMPLE 16

### Effects of SRIF and Peptide SEQ ID NO:1 in Gilts

This experiment was designed to measure the effects of vaccinating pregnant gilts with FEEDMIZA by the subcutaneous route during first pregnancy. Opportunity was taken to compare any response to FEEDMIZA with response to a SRIF-conjugate vaccine delivered in the same delivery vehicle as peptide No.1. For that comparison the quantity of antigen was standardised at 100 μg of SRIF in the SRIF-conjugate, and 100 μg of the FEEDMIZA antigen.

Protocol

Twenty four Landrace X Large White female hybrid gilts were selected, mated and held in conventional dry sow stalls. They were randomly assigned to 3 treatment groups, viz:

| | |
|---|---|
| Group A | non-immunised controls |
| Group B | immunised with FEEDMIZA |
| Group C | immunised with a SRIF-conjugate |

The gilts were immunised by subcutaneous injection of 3 ml of the relevant antigen at the neck-on the day of mating.

The animals were held in conventional single dry sow stalls. At 30 days of pregnancy the treatment groups were reduced to 5 confirmed pregnant animals per treatment group. Subsequent vaccinations were performed at weeks 6, 9 and 12 of pregnancy. Each gilt was fed 3 kg of feed (Breedmore sow ration, Barastoc, Melbourne) each day from mating until farrowing. Each animal was weighed at mating, and at intervals of 3 weeks until farrowing.

Results

No untoward effects were noted with any of the immunisations, either by way of tissue reactions at the site of injection, or general effects on health or behaviour. The pigs which had been immunised with the FEEDMIZA were substantially larger and were especially taller by mid-pregnancy.

The mean weights and standard deviations for the different treatment groups are presented in FIG. 11. There were no stastistical differences between any of the groups up until 12 weeks of pregnancy. At the 12 and 15 weeks, animals in group B was stastically significantly heavier (P<0.05) than the non-immunised and the SRIF-immunised groups. There was a trend for the SRIF-immunised group to be heavier than the non-immunised sows from 9 weeks of pregnancy, but at no point was this difference ever stastically significant (larger numbers of animals may have detected a stastistical difference)

At parturition the non-immunised sows had gained an average of 60 kg per head, had eaten 115×3 kg of feed (345 Kg) and had converted that feed into liveweight gain at a ratio of 5.75:1. The FEEDMIZA-immunised sows had likewise consumed 345 Kg of feed, but had gained an average of 138 kg each during pregnancy, at an efficiency ratio of 2.5:1. The SRIF-immunised group had gained an average of 75 kg per head during the test period at an efficiency of conversion of feed to liveweight gain ratio of 4.6:1.

Discussion and Conclusions

SRIF-conjugate vaccine was not successful in modulating growth stastistically, but the FEEDMIZA preparation significantly improved liveweight gain. It is clear that the FEEDMIZA antigen and the SRIF-conjugate antigens are very different in the effect which they have on the modulation of growth of gestating sows.

Sows immunised with FEEDMIZA preparation were substantially better converters of feed to liveweight gain than were the SRIF immunised and the non-immunised sows.

This experiment has shown that antibodies which are actively generated to FEEDMIZA in gestating female pigs yet to reach mature body weight induce modification to rates of liveweight gain, and feed conversion ratios. It is noted that the pigs were fed measured quantities of feed on a daily basis, so no effect of these antibodies on voluntary feed intake would be influencing the results observed. The modifications must come from either improved digestion, or absorption, or utilisation of the absorbed nutrients.

The four immunisations provided to these sows are probably more than is necessary to achieve successful immunomodulation. The regime was selected to ensure high levels of antibody were present in the animals at all times since the objective was to measure the efficacy of antibodies in modulating the performance. A more practical regime might be vaccination at selection, at confirmation of pregnancy and at transfer to the farrowing house. Further testing may indicate that less than 3 vaccinations per pregnancy produces acceptable results.

### EXAMPLE 17

### Effects of SRIF and Peptide SEQ ID NO:1 on Piglets

This experiment was a continuation of the trial of Example 16. It sought to observe the rate of liveweight gain of piglets during the lactation period up to weaning for piglets whose dams were immunised during gestation with FEEDMIZA, or with a SRIF-conjugate vaccine, using piglets being reared by non-immunised dams as controls.

Protocol

Sows from Example 16 continued to be reared without any further immunisations or manipulations, with the exception that;

a) daily feed offered was increased to 6 kg per head from farrowing to weaning 21 days later.

b) 3 days after farrowing the number of piglets on each sow was reduced to 8 to ensure that each litter of pigs in the experiment was benefiting from the combined effects of the quantity of milk produced, and ingested any antibodies that might be in them, without confounding influences of varying litter sizes, social interactions, and competition for teats. Sows were weighed at weaning.

Piglets were individually identified at birth, and were weighed at birth, and each 7 days until weaning at 21 days of age. All piglets received a routine oral dose of broad spectrum antibiotic (Tribrissen Piglet Suspension, Intervet, Melbourne) at three days of age, as well as an intramuscular supplemental iron injection Pignaemia, Intervet, Melbourne)

Water was available ad libitum to sows and piglets at all times.

Results

The mean liveweights of the non-immunised sows fell by 10.8 kg during the three weeks lactation period, compared with a similar 12 kg for the sows which had been immunised during pregnancy with the SRIF-conjugate vaccine. There was a stastically significant (P<0.05) mean loss of 33.8 Kg for the group which had been immunised during pregnancy with the FEEDMIZ as shown in FIG. 12.

The weights of the sows at farrowing and at weaning were:

| | Weaning | Farrowing | Weight |
|---|---|---|---|
| | weight (kg) | weight (kg) | loss |
| Non-immunised sows | 157.6 ± 24.2 | 146.8 ± 33.3 | 6.8 |
| SRIF-immunised sows | 166.4 ± 26 | 154 ± 27.68 | 7.2 |
| FEEDMIZA-immunised sows | 219.8 ± 20.44 | 186 ± 18.05 | 15.4 |

The results are also represented in FIGS. 13a and 13b.

Sows which were immunised with the FEEDMIZA vaccine were noted to have prolific milk production as evidenced by teats frequently leaking copious quantities of milk, especially during the first week of lactation.

Discussion and Conclusions

Sows lost weight during the lactation phase irrespective of whether they had been vaccinated during pregnancy, or not. This is obviously a response to the demands of lactation which are not adequately met from the increased daily ration offered. However, the magnitude of the loss of weight of sows during lactation which had been immunised with FEEDMIZA during gestation, and which had so dramatically increased in liveweight during pregnancy as reported in the previous trial, was double that which was observed for either the non-immunised or SRIF-conjugate immunised sows. This is a dramatically different pattern from that observed with either of the two other treatment regimes as shown in FIG. 13. One possible explanation for this dramatic loss of weight was that additional stimulation of lactation had been initiated by the anti-FEEDMIZA antibodies acting at the pituitary or mammary glands, or by suppression of normally present inhibitory mechanisms, or both. It was not possible in this experiment to measure the milk production of these sows, but observation of the udders suggested that large quantities of milk were being produced. Parallel studies in sheep have confirmed that in that species, immunisation with FEEDMIZA during pregnancy was associated with a measured 20% increase in output of milk during lactation. It is probable that immunisation with FEEDMIZA during pregnancy resulted in the generation of antibodies in the animal for the 3 week period from farrowing to weaning, and were responsible, via direct or indirect means, for stimulating increased milk production, thereby creating physiological demand upon the sow to repartition nutrients from retained reserves into milk production.

It appears that the FEEDMIZA antibodies are associated with improving the protential productive (liveweight gain or milk production) characteristics of pregnant/lactating pigs. Since both of these functions involve the metabolism of calcium, carbohydrate and protein in particular, these results indicate that antibodies produced to the SRIF-conjugate were less potent in the terms of promoting liveweight gain than were the anti-FEEDMIZA antibodies. It is hypothesised that immunisation with FEEDMIZA into gestating pigs results in increased efficiency of the normal metabolic pathways involving these key metabolites. Whether the effect of the antibodies is stimulatory, or whether it occurs by removal of normal inhibitory mechanisms is not clear.

### EXAMPLE 18

### Effects of SRIF and Peptide SEQ ID NO:1 Following Immunisation of Pre-Farrowing Sows

This experiment sought to study the effects pre-farrowing immunisation of sows with SRIF-conjugate vaccine, or FEEDMIZA vaccine on the subsequent patterns of liveweight gain of their sucking piglets to weaning at 3 weeks of age, and for the post-weaning period The experiment monitored the piglets which were the subject of the previous experiment.

Protocol

The piglets (8 per litter) on each of the 5 sows in the treatment groups (non-immunised, SRIF-conjugate immunised and FEEDMZA-immunised) were individually eartagged for identification, and were weighed at birth, 7, 14, 21 days (the day of weaning) and 35 days of age (2 weeks post-weaning).

During lactation the piglets had ready access to a creep area which was warmed by an infra-red lamp, water ad libitum and creep feed granules (Barastoc, Melbourne) for the period of 18-21 days of age. They had no access to their mothers feed during the period of lactation

At weaning each litter of piglets was placed in a flatdeck weaning cage with 50% of the floor being bedding area, and 50% woven wire mesh. The cage was provided with a small silo feeder containing the same creep/weaner feed which had been offered to the piglets for the 3 days prior to weaning. Feed and fresh drinking water were offered ad libitum.

Results

The mean birthweights of the piglets were:

| | |
|---|---|
| i) | born to non-immunised sows |
| | 1.22 ± 0.61 Kg |
| ii) | born to SRIF-immunised sows |
| | 1.40 ± 0.29 Kg |
| iii) | born to FEEDMIZA-immunised sows |
| | 1.58 ± 0.21 Kg |

The piglets born to the FEEDMIZA-immunised sows were stastistically heavier (P<0.05) than those born to the other groups (which were not stastistically different from each other).

The liveweights of the piglets at weaning were:

| | |
|---|---|
| i) | piglets sucking from non-immunised sows |
| | 6.05 ± 0.63 Kg |
| ii) | piglets sucking from SRIF-immunised sows |
| | 7.32 ± 1.30 Kg |
| iii) | piglets sucking from FEEDMIZA-immunised sows |
| | 8.30 ± 1.13 Kg |

The differences in the mean weights between each treatment group was stastistically significant (P<0.05).

Liveweights of the piglets 2 weeks after weaning were:

| | |
|---|---|
| i) | piglets which had been sucking non-immunised sows |
| | 7.58 ± 0.66 Kg |
| ii) | piglets which had been sucking SRIF-immunised sows |
| | 8.89 ± 1.33 Kg |
| ii) | piglets which had been sucking FEEDMIZA-immunised sows |
| | 10.62 ± 0.81 Kg |

Each of these mean livweights was stastistically different from each other (P<0.05).

The mean liveweights of each of the treatment groups of piglets is presented in FIG. 14.

Discussion and Conclusions

Immunisation of pregnant sows with SRIF-conjugate antigen, or with FEEDMIZA resulted in increased rates of liveweight gain of piglets sucking those dams compared with non-immunised sows. This suggests that immunomanipulation of animals for the purpose of increasing milk production is possible. Immunisation of pregnant sows with FEEDMIZA vaccine produced stastistically significanly heavier weights of piglets which sucked them for 3 weeks than was observed for piglets sucking either non-immunised, or SRIF-immunised sows at the observation points of birth, weaning and 2 weeks post-weaning. This observation suggests that the process of immunomanipulation had been more effectively achieved with FEEDMIZA than with the SRIF-conjugate.

### EXAMPLE 19

### Administration of Peptide SEQ ID NO:1 to Sheep

Sheep have been the subject of numerous published attempts at immunomodulation with a variety of hormones. Sheep have many production characteristics that are similar to the pig production cycle, viz a relatively short period of gestation, rapid rate of gain of the sucking young, and a relatively short period of lactation. They are relatively easy animals for measuring milk production, and they lend themselves readily to blood collection for measurement of antibodies, circulating hormones or metabolites.

The following experiments compared responses of FEEDMIZA-immunised animals with non-immunised control animals.

Since stimulation of milk let-down (and therefore measurable milk production) is dependent upon there being good vigorous sucking, and complete offtake of what milk is produced, it is desirable to use ewes with twin lambs. Studies involving the measurement of milk production were performed on a known high twinning line of mature aged ewes, ensuring that adequate numbers of twin-bearing ewes would be available for the experiments.

Experiment 1

This experiment was designed to measure the effect of immunising pregnant merino ewes three times during pregnancy, and comparing the weights of the ewes at first immunisation, at lambing and at “weaning” 6 weeks after lambing with comparable ewes and lambs that have not been immunised.

Protocol

Twenty merino ewes that had been mated previously following synchronisation with intra-vaginal sponges (Repromap; Upjohn, Australia) were given an intramuscular injection of pregnant mare serum gonadotropin (Folligon, Intervet, Melbourne, Australia). When the sponges were withdrawn, the animals were randomly allocated into two groups of 10 each (groups to be immunised and to remain non-immunised) at 60 days of pregnancy. The animals were proven to be pregnant with twin lambs using an ultrasonic scanner.

The pregnant ewes were maintained on pasture during pregnancy as a single mob of sheep. Free choice was provided to lucerne hay during the last four weeks of pregnancy. Immediately after the sheep lambed they were moved indoors and offered 2.5 Kg per day of a 4:1 mixture of lucerne chaff and rolled barley until 6 weeks of age, at which point the experiment was concluded and the ewes and lambs were turned out to pasture.

The ewes which were to be immunised were injected with 3 ml of FEEDMIZA subcutaneously in the flank at 90, 110 and 132 days of pregnancy.

The liveweights of the ewes were recorded at the time of first immunisation, at birth and at 6 weeks of age “weaning”. Liveweights of the lambs were recorded at birth and at weaning.

Wool samples (100 square centimetres) were collected as mid side patches as described by Wynn et al (1988) at time of first immunisation, lambing and weaning of the ewes, and at weaning for the lambs. The follicle density and types were assessed by histological examination of core samples collected at birth, 1 week of age and at weaning.

Milk production was measured one day of each week for the first 6 weeks of lactation by depriving the lambs access to their mothers for a period of 6 hours. After the 6 hours of lamb deprivation the ewes were handmilked with the total weight of milk being recorded. A 10 ml sample of this milk was taken for assay, and the remainder was fed to the lambs by hand using teats attached to the pen draining a pouch containing the milk.

Results

The weights of the ewes at first immunisation were 57.3±2.4 kg for the group to be immunised, and 57.5±2.7 kg for the group to remain as non-immunised controls. At birth the immunised ewes weighed 53.2±3.0 kg while the non-immunised group weighed 51.8±2.7 kg. At weaning the immunised ewes weighed 55.2±3.1 kg and the non-immunised group weighed 52.7 kg. There were no stastistically significant differences between the weights of the ewes at any stage of the experiment, nor was there any significant differences in the birthweights of the lambs. (4.4±0.11 kg for the immunised groups compared with 4.4±0.08 kg for those in the non-immunised group). The weights of the lambs at weaning were 6.72±0.8 kg in the non-immunised ewes, and 8.07±1.0 kg in the immunised ewes, as shown in FIG. 15. This difference in liveweights was stastistically significant (P<0.05).

The milk yields recorded for the ewes are presented in FIG. 16 and show that the milk production of the immunised ewes was approximately 20% greater at each observation.

There was no stastistically significant differences in the milk fat, milk lactose or milk protein levels measured in the samples of milk collected from the treatment groups at each milk collection. There were slight differences in the analyses from week to week.

The yields of wool were approximately 10% heavier from the immunised ewes than from the non-immunised ewes during pregnancy, and during lactation. Yields of wool from the lambs consuming milk from the immunised ewes was 20% greater than that produced by the lambs drinking milk from the non-immunised ewes. In addition the wool which was harvested from the lambs from the immunised ewes was significantly finer than that which was observed from the non-immunised lambs. The lambs on the immunised ewes had greater densities of secondary and primary follicles.

Discussion and Conclusions

Immunisation of pregnant ewes with FEEDMIZA resulted in lambs which grew at a quicker rate of liveweight gain than was observed with the lambs produced by the non-immunised ewes. This was apparently associated with a greater quantity of milk produced by the ewes. The milk produced by the immunised ewes was of normal analysis and therefore of equal nutritional value compared with milk from non-immunised ewes. Immunised ewes produced more clean scoured wool during pregnancy and lactation, suggesting that there had been an effective improvement in nutritional status of the immunised ewes during the period of gestation (improved wool production) and during lactation (improved wool and milk production). These favourable alterations to productivity are consistent with antibodies produced by the immunised ewes exerting their effects upon wool follicle and lactogenic mammary cells.

The increased growth rates of lambs that were raised by the immunised ewes could be explained in that these lambs had more milk available to them, so presumably, they drank more milk. As this milk was of normal analysis, these lambs took in more nutrients and were consequently receiving a higher plane of nutrition. This may not be the complete explanation since the colostrum and milk are also heavily laden with antibodies to FEEDMIZA. These antibodies may have moderating effects on feed utilisation by the lambs.

Of enormous potential commercial significance for merino sheep is the observation that the lambs raised by the immunised ewes showed markedly increased densities of wool follicles in the skin. This change would be one that persisted for life. Even more significantly the ratio of secondary to primary follicles was improved in favour of the secondary follicles, which are those which have been reported to be associated with production of the fine wool fibres. This would suggest that these lambs have been permanently modified in favour of the production of more wool of a finer quality simply because they have been consuming large quantities of milk containing anti-FEEDMIZA antibodies for the first 6 weeks of life.

### EXAMPLE 20

### Administration of Peptide SEQ ID NO:1 to Cattle

This experiment was designed as a simple test to see if vaccinating range-bred and reared-beef cattle would result in heavier liveweights during pregnancy. A further observation was to be made of the growth of calves sucking cows which were immunised with FEEDMIZA during gestation.

Protocol

A line of fifty Hereford×Angus heifers was selected and placed onto actively growing pasture. The heifers were oestrus synchronised by injecting with Lutalyse (Upjohn, Rydalmere, Australia) and naturally mated to Hereford bulls. The animals were randomly divided into two groups at the time of synchronisation; one group of 20 animals was to be the immunised group, and a second group of 20 animals was the non-immunised heifers.

Animals to be immunised were injected with 3 ml of emulsion subcutaneously in the neck at mating, 3 months of pregnancy and two weeks prior to parturition.

The animals were weighed at the time of mating, at parturition and at 3 months after parturition. The calves produced were weighed at birth and each 28 days thereafter until 3 months old.

Results

The mean weights of the heifers at mating were 360±8.5 kg for the non-immunised group, and 355±12.8 kg for the group to be immunised.

At parturition the mean weights of the non-immunised control animals was 385±15.5 kg, and for the immunised group was 440 kg for the immunised cows. (an average difference of 22%) The mean weights of the cows 3 months after parturition were 375±20.5 kg or the non-immunised animals, and 415±24.5 kg or the immunised cows ( an average difference of 10.6%) Results are presented in FIG. 17.

Mean birth weight of the non-immunised calves was 35±3.5 kg compared with 40±4.5 kg. (an average difference of 14%). Mean weights of the calves three months after birth were 116.2±6.4 kg for the non-immunised control group, and 145.0±7.5 kg for the immunised group (an average difference of 20%). The average gain of the non-immunised calves for days 1-84 was 81.2 kg, and for the immunised group was 105 kg (an average difference of 23.8 kg, or 29.3%), as shown in FIG. 18.

Total net gain of liveweight (cow plus calf) from mating to 84 days after calving was 131.2 Kg for the non-immunised group, and 205 Kg for the immunised group.

Discussion and Conclusions

Immunisation of range-bred and range-reared beef heifers with FEEDMIZA has resulted in the initiation of greater weight gains of these animals during pregnancy, and in the production of heavier calves at birth. The calves born to immunised or to non-immunised cows were both approximately 9% of the weight of the cow at birth. Calves sucking the vaccinated dams grew at a more rapid rate for the first 84 days after birth. This is a similar pattern to that which has been observed following immunisation of pregnant sows or pregnant ewes, and provides further circumstantial evidence that FEEDMIZA has the potential to favourably alter the efficiency of beef breeding.

### EXAMPLE 21

### Effects of Different Types of Adjuvants

Lambs were immunised at 35 kg liveweight with 0.5 mg of SRIF conjugated to BSA (bovine serum albumin) in a variety of delivery vehicles (adjuvants) at day 0, 21 and 42 (n=6 animals per group).

The growth responses measured over an 84 day period were:

| | |
|---|---|
| non immunised controls | 2.0 ± 1.37 kg |
| FCA immunised | 4.5 ± 1.60 kg |
| MDP immunised | 7.0 ± 1.3 kg |
| FIA & MDP immunised | 5.0 ± 1.3 kg |
| DEAE immunised | 3.5 ± 0.6 kg |
| Quill A immunised | 4.0 ± 1.5 kg |
| Oil immunised | 7.5 ± 1.2 kg |

It is concluded that the type of adjuvant used may have a baring upon the efficacy of antibodies generated.

A significant response was observed when the oil of the invention was used as the adjuvant. Immunised animals did not show any evidence of abscess formation, and indicates that it is a potential commercial preparation. Other materials which could conceivably be used as adjuvants, or additions to delivery vehicles to act as adjuvants are aluminum salts, lipopolysaccharides, sorbitan trioleate, Pluronics, Tetronics, squalene, liposomes, immunostimulatory complex, cholera toxin, heat labile toxin of E. coli, interleukins or the like.

### EXAMPLE 22

### Effects of Variations to Antigen Structure

The affinities of antibodies to SRIF, the cyclosised form of the preferred sequence (SEQ ID Nos 2 to 3) and the linear form (SEQ ID No. 4) were determined for hyperimmune sheep and swine-sera produced against each of the three presentations.

Antibodies Affinities of Antisera for Somatostatin

Affinities of the antibodies for somatostatin were established using Scatchard plot analysis as described previously by Holst J J, Jorgensen P N, Rasmussen T N & Schmidt P (1992), “Somatostatin restraint of gastrin secretion in pigs revealed by monoclonal antibody immunoneutralization”. American journal of Physiology 263:G908-G912. Affinites for sheep and swine antibodies which recognise and bind somatostatin are summarised below:

| ANTIGEN | ANTIBODY | AFFINITY FOR SOMATOSTATIN | |
|---|---|---|---|
| Anti-SRIF | sheep | 1.5 × 10¹¹± 1.2 × 10¹ | 1 mol⁻¹ |
| Anti-SRIF | swine | 2.0 × 10¹⁰± 1.4 × 10¹ | 1 mol⁻¹ |
| Cyclosised | sheep | 1.9 × 10⁸ ± 2.2 × 10¹ | 1 mol⁻¹ |
| Cyclosised | swine | 3.0 × 10⁸ ± 1.0 × 10¹ | 1 mol⁻¹ |
| Linear | sheep | 1.3 × 10⁷ ± 0.2 × 10² | 1 mol⁻¹ |
| Linear | swine | 1.4 × 10⁷ ± 1.6 × 10¹ | 1 mol⁻¹ |

It is evident from these data that the anti-SRIF antisera raised in sheep and swine gave rise to a much higher association constant than antisera raised in animals immunised with Cyclosised or the linear equivalent of the construct.

Antibody studies with SRIF and glucagon reveal that some cross-over effect occurs between both molecules. The structure of SRIF contains the sequence S-T-F-T which is homologus with a portion of the glucagon molecule. Antibodies raised to the preferred peptide have no cross reactivity with glucagon.

Pregnant pigs which were immunized twice in the last trimester of pregnancy with SEQ ID Nos 3, 4, 5 and SRIF 14 (native somatostatin) conjugate presented with the preferred oil all produced anti-SRIF antibodies in colostrum and milk of the sow and in the blood and gut scrapings of the piglets at 3 days and 21 days of age.

The absolute growth rates of the piglets from birth to weaning were:

Piglets from non-immunised 165 g per day n=40 control sows

Piglets from sows immunised 275 g per day n=42 with SEQ ID No:3

Piglets from sows immunised 240 g per day n=44 with SEQ ID No:4

Piglets from sows immunised 180 g per day n=42 with SRIF conjugate

### EXAMPLE 23

### Effects of SEQ ID No 3 in Piglets

The type of antibody produced in response to the cyclic peptide delivered in the preferred oil has a bearing upon the growth of the immunised animals compared to SRIF 14 conjugate delivered in the same manner.

Sows immunized twice in the last trimester of pregnancy with SEQ ID No 3 produced piglets which were on average 27% heavier at weaning (at 21 days of age) than the offsprings of sows immunised with SRIF 14 conjugate vaccine. The levels of anti-SRIF antibodies measured are given below:

| TREATMENT | DAY 3 | DAY 21 |
|---|---|---|
| Gut IgG | | |
| (OD @ 405 nm ± SD) | | |
| SRIF/BSA | 0.684 ± 0.548 | 0.259 ± 0.18 |
| SEQ ID NO:3 | 0.51 ± 0.21 | 0.123 ± 0.09 |
| Gut IgA | | |
| SRIF/BSA | 0.189 ± 0.133 | 0.037 ± 0.05 |
| SEQ ID NO:3 | 0.12 ± 0.157 | 0.10 ± 0.087 |
| Plasma IgG | | |
| SRIF/BSA | 1.38 ± 0.44 | 1.779 ± 0.13 |
| SEQ ID NO:3 | 0.9 ± 0.13 | 2.12 ± 0.23 |
| Plasma IgA | | |
| SRIF/BSA | 1.067 ± 0.469 | 0.798 ± 0.49 |
| SEQ ID NO:3 | 1.43 ± 0.217 | 0.567 ± 0.21 |
| Sow's milk IgG | | |
| SRIF/BSA | 1.6 ± 0.22 | |
| SEQ ID NO:3 | 1.4 ± 0.162 | |
| Milk/colostrum IgA | | |
| SRIF/BSA | 0.029 ± 0.035 | |
| SEQ ID NO:3 | 0.026 ± 0.028 | |

Significant quantities of antibodies to SRIF were detected in the plasma and gut scrapings of piglets sucking sows immunised with SRIF/BSA or SEQ ID NO:3 emulsified in the oil of the invention.

The results indicate that although the amount of antibody to SRIF, measured in colostrum, milk, gut scrapings or piglet serum up to weaning were similar, the functionality of the antibodies was different, as evidenced by the difference in growth rates observed.

The peptides when administered (via intramuscular, subcutaneous, intramammary, oral or intraperitoneal delivery) to the vaccine recipient in conjunction with or in the absence of any adjuvant or immunostimulatory system, will elicit specific antibodies which alter directly or indirectly the endocrine systems associated with digestion and subsequent metabolism of nutrients. However, the preferred form of antigen delivery involves the ilumunogens being mixed, suspended or preferably emulsified in a non-macrophage stimulating system injected either subcutaneously or intraperitoneally. An important feature of the preferred delivery system is that it facilitates the expression of the humoral immune response to multiple antigens as if each was presented alone. Furthermore, since the delivery system elicits predominantly a humoral response, without involving macrophage stimulation, the antibodies are extremely potent (high titres and affinity) and involve the whole complement of isotypes, especially those associated with mucosal surfaces (IgA and IgM).

The mechanisms by which the invention improves animal productivity are extremely complex and not understood completely. However, it has been established that the antibodies alter the metabolism of particularly somatostatin, gastrin, insulin, and glucogon; and augment the circulating concentrations of insulin-like growth factor (IGF-I and II). The endocrine ramifications associated with altering the metabolism of these hormones are vast and include changes in the circulating concentrations of, at least, gastrin, cholecystokinin, motilin, secretin, the thyroid hormones, glucagon, insulin, IGF I and II, somatostatin, prostaglandins, histamine, and vasoactive intestinal peptide. It has been demonstrated from the results obtained from our laboratory that immunisation against the peptides induce changes in both gastrointestinal and metabolic functions. For example the rate of the secretion of gastric acid and the activity of many of the gastric proteases are retarded significantly in response to both chemical and physiological stimulation following immunisation. In addition, the motility of digest a through various segments of the gastrointestinal tract are altered significantly and consequently the absorption and metabolism of key metabolites are enhanced. Furthermore, the antibodies allow many cells particularly those associated with the endocrine system to be more responsive to Ca²⁺ ions, thus increasingly the secretion of hormones associated with the somatotropic axis and gastrointestinal tract. There is an improved capacity for cells to perform optimally even though Ca²⁺ levels are suboptimal. The effect of this is that mRNA production within cells, especially muscle fibres, mammotrophs and somatotrophs are enhanced.

It is postulated that the combination of events observed in response to immunisation enhance digestion and absorption of feedstuffs and subsequent metabolism of key metabolites leading to improve productive capacities of immunised animals.

Although the invention has been described in detail for the purposes of clarity, it will be understood that various modifications may be made by a person skilled in the art without departing from the scope of the invention.

### Table 2

Birth weights of piglets from sows immunised during pregnancy with either SSTR 1, SSTR 2, SSTR 3, SSTR 4 and SSTR 5 antigens or placebo injections

| TREATMENT | BIRTHWEIGHT | |
|---|---|---|
| SSTR 1 | 1.5 ± 0.2 | k |
| SSTR 2 | 1.65 ± 0.1 | kg |
| SSTR 3 | 1.7 ± 0.15 | kg |
| SSTR 4 | 1.5 ± 0.2 | kg |
| SSTR 5 | 1.7 ± 0.1 | kg |
| Control | 1.35 ± 0.2 | kg |

TABLE 3
Mean titres of anti-SSTR antibodies in the colostrum if immunised sows
and in the plasma and gut scrapings of sucking piglets at 21 days of age

| SAMPLE | CONTROL | SSTR 1 | SSTR 2 | SSTR 3 | SSTR 4 | SSTR 5 |
|---|---|---|---|---|---|---|
| Plasma | 0 | 3025 | 5412 | 2564 | 1248 | 6458 |
| Gut | 0 | 2000 | 1895 | 3654 | 1478 | 4785 |
| Colostrum | 0 | 5002 | 8425 | 7845 | 5784 | 8563 |

TABLE 4
Mean numbers of primary and secondary follicles of lambs
sucking ewes immunised against SSTR 2, 3 and or placebo
injections at birth and 6 weeks of age.

| AGE | MEASUREMENT | CONTROL | IMMUNISED |
|---|---|---|---|
| | Secodary follicle | 57 | 67 |
| B | Primary follicle | 13 | 17 |
| i | Total follicle | 70 | 84 |
| r | Secondary density | 36 cm⁻² | 38 cm⁻² |
| t | Primary density | 8 cm⁻² | 10 cm⁻² |
| h | Total density | 44 cm⁻² | 48 cm⁻² |
| | Ratio S/P | 5 | 4.5 |
| 6 | Secodary follicle | 95 | 118 |
| | Primary follicle | 5 | 10 |
| W | Total follicle | 100 | 128 |
| e | Secondary density | 55 | 70 |
| e | Primary density | 3 | 4 |
| k | Total density | 58 | 74 |
| s | Ratio S/P | 17 | 20 |

TABLE 5
Liveweight gains and average daily liveweight gain of
female pigs immunised with FEEDMIZA by various routes
of injection, and placebo injected pigs. (males)

| Treatment | | |
|---|---|---|
| (kg) | n | Weight at 21 weeks |
| Placebo injected | 12 | 583 ± 24.1 |
| 90.8 ± 3.21 | | |
| Subcutaneous immunisation | 12 | 650 ± 24.2 |
| 110.5 ± 3.22 | | |
| Intraperitoneal immunisation | 12 | 625 ± 21.0 |
| 104 ± 2.79 | | |
| Immunised intramuscularly | 12 | 600 ± 19.6 |
| 96 ± 2.61 | | |

The heaviest group was those which had been immunised subcutaneously. They were significantly heavier than all other treatment groups (p<0.05). The pigs immunised by the intraperitoneal route were significantly heavier than those immunised intramuscularly and those given placebo injections (p<0.05). There was no significant difference between the intramuscularly immunised and the placebo groups.

TABLE 6
Liveweight gains and average daily gains for female pigs
immunised with FEEDMIZA by varying routes of injection,
and placebo injected pigs.

| | n | |
|---|---|---|
| Treatment | Average daily gain | |
| (kg) | (grams) | Weight at 21 weeks |
| Placebo injected | | |
| 92 ± 2.49 | 12 | 575 ± 18.79 |
| Immunised subcutaneously | | |
| 104 ± 2.79 | 12 | 650 ± 21.05 |
| Immunised intraperitoneally | | |
| 9.5 ± 2.1 | 12 | 597 ± 15.79 |
| Immunised intramuscularly | | |
| 98.7 ± 2.49 | 12 | 617 ± 18.79 |

Heaviest liveweight was recorded in the group which had been immunised by the subcutaneous route. This was significantly heavier (p<0.05) than t he weight of the group immunised intramuscularly. The weights of both of these groups were significantly heavier (p<0.05) than the placebo injected group. There were no significant differences between the weights of the other treatments.

References

Cohick, W. S. and Clemmons, D. R. Annual ana Review of Physiology, 1993 55 131-153.

Crowe, M. A., Enright, W. J., and Roche, J. F. Journal of Animal Science, 1995 73 2406-2417.

Dockray, G. J., and Taylor, I. L. Gastroenterology, 1976 71 971-977.

Hoskinson, R. M., Rigby, R. D. G., Mattner, P. E., Huynh, V. L., D'Occhio, M., Neish, A., Trigg, T. E., Moss, B. A., Lindsey, M. J., Coleman, G. D., and Schwartzkoff, C. L. Australian Journal of Biotechnology, 1990 4 166-176.

Kamoi, K., Hama, H., Ito, S., and Matsuoka, M. Endocrinologia Japonica, 1977 24 239-243.

Kaushansky, A., Bauminger, S., Koch, Y., and Lindner, H. R. Acta Endocrinologica, 1977 84 795-803.

Lindstedt, G. European Journal of Endocrinology, 1994 130 429-432.

Melmed, S., Harada, A., Hershman, J. M., Krishnamurthy, G. T. and Blahd, W. H. Journal of Clinical Endocrinology and Metabolism, 1980 51 358-363.

Meloen, R. H. Livestock Production Science, 1995 42 135-145.

Moore, K. L., Armstrong, J. D., Harvey, R. W., Campbell, R. M., and Heimer, E. P Domestic Animal Endocrinology, 1992 9 125-139.

Patel, Y. C. In “Basic and Clinical Aspects of Neuroscience., Vol 4. Somatostatin” (C. Weil, E. E., Müller and M. O. Thorner, eds), 1992 1-16 Springer Verlag, Berlin.

Pekas, J. C. and Trout, W. E. Journal of Animal Science, 1993 71 2499-2505.

Pell, J. M. and Aston, R. Livestock Production Science, 1995 42 123-133.

Pell, J. M. and James, S Journal of Endocrinology, 1995 146 535-541.

Reisine, T. and Bell, G. I. Endocrine Reviews, 1995 16 427-442.

Scanlon, A. R., Sunderland, S. J., Martin, T. L., Goulding, D., O'Callaghan, D., Williams, D. H., Headon, D. R., Boland, M. P., Ireland, J. J. and Roche, J. F Journal of Reproduction and Fertility, 1993 97 213-222.

Scaramuzzi, R. J., Davidson, W. G., and Van Look, P. F. A. Nature, 1997 269 807-808.

Thomson, D. L., Jr, Southern, L. L., St. George, R. L., Jones, L. S., and Garza, F., Jr Journal of Animal Science, 1985 61 1498-1504.

Westbrook, S. L., Chandler, K. D. and McDowell, G. H. Australian Journal of Agricultural Research, 1993 44 229-238.

Wynn, P. C., Shahneh, A. Z., Rigby, R. D. G., Behrendt, R., Giles, L. R., Gooden, J. M., and Jones, M. R Livestock Production Science, 1995 42 247-254.

Wynn, P. C., Wallace, A. L. C., Kirby, A. C., Annison, E. F. Effects of growth hormone administration on wool growth of Marino sheep. Australian Journal of Biological Sciences, 41 177-187.

1. A non-naturally occurring peptide with an amino acid sequence which is derived from, or is similar to a native animal hormone, carrier protein, binding protein or receptor for said hormone, wherein said peptide is capable of eliciting one or more antibodies which are able to modulate the activity of said hormone or receptor in vivo.

2. A peptide according to claim 1, wherein the native animal hormone is somatostatin.

3. A peptide according to claim 1 or claim 2, selected from the group consisting of one or more of peptide Nos. 1 to 53 as herein defined.

4. A peptide according to claim 3, wherein the peptide is a peptide selected from the group consisting of SEQ ID NO:1, SEQ ID NO:2, SEQ ID NO:3 and SEQ ID NO:51.

5. A peptide according to any one of claims 2 to 4, wherein the antibodies modulate the activity of one or more hormone receptor selected from the group consisting of SSTR2, SSTR3 and SSTR5.

6. A peptide according to claim 1, wherein the binding protein is insulin-like growth factor binding protein (IGFBP).

7. A peptide according to claim 6, selected from the group consisting of peptide Nos. 54 to 57.

8. A peptide according to any one of claims 1 to 7, wherein the hormone, carrier protein binding protein or receptor therefor is of human origin.

9. A peptide according to any one of claims 1 to 8, coupled to a carrier to form a peptide/carrier complex.

10. A peptide according to claim 9, wherein the carrier is a multiple antigen peptide (MAP) system.

11. A peptide according to claim 10, wherein the MAP system comprises an oligomeric branching lysine core.

12. A peptide according to claim 11, wherein the MAP system comprises at least 18 branching lysine core.

13. An immunologically reactive molecule (IRM) which is specific for the peptide of any one of claims 1 to 12.

14. An immunologically reactive molecule according to claim 13, which molecule is selected from the group cosisting of naturally occurring antibodies, recombinant antibodies, scantibodies, synthetic antibodies, fusion antibodies, chimeric antibodies and functional fragments thereof.

15. A pharmaceutical composition comprising an immunogenically effective amount of the peptide or molecule according to any one of claims 1 to 14, together with a pharmaceutically or veterinarily acceptable carrier.

16. A vaccine preparation comprising the peptide or molecule according to any one of claims 1 to 15.

17. A veterinarily or pharmaceutically acceptable carrier, comprising shark oil which has immuno-adjuvant activity.

18. A carrier according to claim 17, wherein said oil is from deep sea sharks and stimulates antibody production in an epithelial or mucosal surface.

19. A carrier according to claim 17 or claim 18, wherein said oil comprises 30-50% diacylglycerol ethers

20. A carrier according to claim 19, comprising:
20-70% octadec-9-enylglyceryl ether
3-25% 1-hexadecylglyceryl ether
1-15% hexadec-7-enylglyceryl ether
1.5-20% octadecyl glyceryl ether
1-15% eicosa-9-enylglyceryl ether
1-25% lecithin
1-25% DL alpha tocopherol acetate
0-3% 1, 2, 5-dihydroxycholecalciferol
0-5% vitamin A
0-40% non-mineral oil.

21. A method of producing an immunogenic composition, comprising the step of contacting a peptide capable of eliciting an immune response with a carrier according to any one of claims 17 to 20.

22. A method according to claim 21, wherein the peptide is a peptide according to any one of claims 1 to 12.

23. A method of delivering to an animal a peptide capable of eliciting an immune response, comprising the step of administering said peptide together with an effective amount of a carrier according to any one of claims 17 to 20.

24. A method of modulating one or more hormonal responses in an animal, comprising the step of administering to said animal a hormone modulating effective amount of a peptide or molecule according to any one of claims 1 to 15.

25. A method according to claim 24, wherein the hormonal response is a response to one or more hormones selected from the group consisting of somatostatin, gastrin, insulin, glucagon, prolactin, molitin, cholecystokinin, secretin, prostaglandins, IGFBP, IGF-I, IGF-II, growth hormone, thyroid hormone, luteinising hormone (LHRM) and hormones of the adrenal gland.

26. A method of enhancing gastrointestinal function in an animal, comprising the step of administering an effective amount of a peptide based on SSTR and/or IGFBP to said animal.

27. A method of increasing anabolism and/or body weight in an animal, comprising the step of administering an effective amount of a peptide based on SSTR and/or IGFBP to said animal.

28. A method of increasing circulating insulin, IGF-I and/or IGF-III in an animal, comprising the step of administering an effective amount of a peptide based on SSTR and/or IGFBP to said animal.

29. A method of suppressing gastric enzymes in an animal, comprising the step of administering an effective amount of a peptide based on SSTR to said animal.

30. A method of increasing fibre production and optionally further altering the proportion of secondary to primary follicles in a fibre producing animal, comprising the step of administering an effective amount of a peptide based on SSTR and/or IGFBP to said animal.

31. A method of increasing milk production in a milk producing animal, comprising the step of administering an effective amount of a peptide based on SSTR and/or IGFBP to said animal.

32. A method of decreasing the activity of the c-fos gene and/or increasing the activity of the c-jun gene in an animal, comprising the step of administering an effective amount of a peptide based on SSTR to said animal.

33. A method of altering calcium metabolism in an animal, comprising the step of administering an effective amount of a peptide based on SSTR to said animal.

34. A method of stimulating an immune response to a hormone, carrier protein, binding protein or a hormone receptor in an animal wherein said immune response modulates hormone activity, said method comprising the step of administering an immune response inducing effective amount of a peptide according to any one of claims 1 to 13.

35. A method of stimulating an antibody response to a hormone, carrier protein, binding protein or hormone receptor, wherein said antibody response modulates hormone or receptor activity in an animal and wherein said antibody response results in antibodies being secreted on the mucosa of the animal and/or in the milk of said animal, said method comprising the step of administering to said animal an antibody stimulating effective amount of the peptide of the invention.

36. A kit comprising a peptide according to any one of claims 1 to 13.

37. A kit comprising a molecule according to claims 13 or 14.

38. A kit according to claim 36 or claim 37, further comprising a shark oil which has immuno-adjuvant activity.
